(19) United States
(12) Patent Application Publication
(10) Pub. No.: US 2026/0041770 A1
(43) Pub. Date: Feb. 12, 2026

(54) CELL MEMBRANE-ATTACHED HYALURONIC ACID-CD44 SPECIFIC BINDING REACTION POLYMER COMPOUND TO ENHANCE THE IMMUNE-ANTICANCER FUNCTION OF NATURAL KILLER CELLS

(71) Applicant: DONGGUK UNIVERSITY INDUSTRY-ACADEMIC COOPERATION FOUNDATION, Seoul (KR)

(72) Inventors: Kyo Bum KIM, Seoul (KR); Sung Jun KIM, Seoul (KR); Chae Eun LEE, Seoul (KR); Hee Won PARK, Seoul (KR); Won Jeong LEE, Pohang-si (KR); Ashok Kumar JANGID, Seoul (KR)

(73) Assignee: DONGGUK UNIVERSITY INDUSTRY-ACADEMIC COOPERATION FOUNDATION, Seoul (KR)

(21) Appl. No.: 19/360,343

(22) Filed: Oct. 16, 2025

Related U.S. Application Data
(63) Continuation of application No. PCT/KR2024/004799, filed on Apr. 11, 2024.
(63) Continuation of application No. PCT/KR2024/004800, filed on Apr. 11, 2024.
(63) Continuation of application No. PCT/KR2024/011486, filed on Aug. 5, 2024.

(30) Foreign Application Priority Data
Apr. 17, 2023 (KR) 10-2023-0050304
Apr. 17, 2023 (KR) 10-2023-0050306
Apr. 17, 2023 (KR) 10-2023-0050311
May 1, 2023 (KR) 10-2023-0056762
Nov. 13, 2023 (KR) 10-2023-0156366
Nov. 13, 2023 (KR) 10-2023-0156371

(51) Int. Cl. A61K 40/42; A61K 40/15; C08G 81/00

(52) U.S. Cl. CPC A61K 40/4223; A61K 40/15; C08G 81/00; A61K 2239/27

(57) ABSTRACT

Provided is a polymer compound comprising: a hydrophobic moiety binding to natural killer (NK) cells; a cancer cell recognition moiety; and a linker, wherein the hydrophobic moiety is bound to one end of the linker and the cancer cell recognition moiety is bound to the other end of the linker and recognizes NK cells and cancer cells, wherein the cancer cell recognition moiety comprises hyaluronic acid.

### CROSS-REFERENCE TO RELATED APPLICATIONS

This is a continuation of International Patent Application PCT/KR2024/004799 (filed 11 Apr. 2024), which claims the benefit of Republic of Korea Patent Application 10-2023-0050304 (filed 17 Apr. 2023), Republic of Korea Patent Application 10-2023-0050306 (filed 17 Apr. 2023), Republic of Korea Patent Application 10-2023-0050311 (filed 17 Apr. 2023), Republic of Korea Patent Application 10-2023-0056762 (filed 1 May 2023), and Republic of Korea Patent Application 10-2023-0156366 (filed 13 Nov. 2023). The entire disclosure of each of the foregoing patent applications is hereby incorporated by reference herein.

This is a continuation of International Patent Application PCT/KR2024/004800 (filed 11 Apr. 2024), which claims the benefit of Republic of Korea Patent Application 10-2023-0050304 (filed 17 Apr. 2023), Republic of Korea Patent Application 10-2023-0050306 (filed 17 Apr. 2023), Republic of Korea Patent Application 10-2023-0050311 (filed 17 Apr. 2023), Republic of Korea Patent Application 10-2023-0056762 (filed 1 May 2023), and Republic of Korea Patent Application 10-2023-0156371 (filed 13 Nov. 2023). The entire disclosure of each of the foregoing patent applications is hereby incorporated by reference herein.

This is a continuation of International Patent Application PCT/KR2024/011486 (filed 5 Aug. 2024), the entire disclosure of which is hereby incorporated by reference herein.

### TECHNICAL FIELD

The present invention relates to a polymer compound which may be synthesized and prepared to be bound to a surface of a natural killer (NK) cell and enhance an anticancer function of the corresponding cell, and may be injected into the body to selectively recognize and kill CD44 cancer cells, thereby dramatically improving a preventive or therapeutic effect on cancer.

### BACKGROUND ART

Immunotherapy is one of the most successful and innovative methods for treating various cancers. Natural killer (NK) cells are special effect cells having a unique ability to identify and remove tumor cells without disadvantages of T cells such as a lack of autologous T cells, an off-target toxicity, and a cytokine release syndrome, and have recently attracted attention as cells capable of replacing T cells.

However, there is a problem in that an ability to selectively target tumor cells is insufficient due to an immunosuppressive tumor microenvironment (TME) and a lack of specific cancer-targeting ligands of NK cells, thus resulting in a decrease in an efficacy of immunotherapy.

Chimeric antigen receptor (CAR)-based genetic engineering is one of the technologies which may overcome a target limitation of NK cells and improve a therapeutic performance, and clinical trials are underway around the world. As interest and demand for CAR-NK cells increase, problems related to CAR-based engineering are also emerging.

Specifically, a process of preparing genetically engineered CAR-NK cells suffers from low infection efficiency, and a unique biological activity of NK cells for tumors may be reduced due to unpredictable mutagenesis. Accordingly, there is a demand for the development of new technologies which do not depend on genetic modification.

As an alternative to overcome limitations associated with a production of CAR-NK cells, surface engineering technology of NK cells is being studied. As a related technique, a technique of targeting CD22 by modifying a surface of NK cells through glycoengineering has been developed, but an expression of a target ligand through glycoengineering is entirely dependent on intracellular metabolism, and thus it is difficult to precisely control the target ligand.

### DISCLOSURE

### Technical Problem

One technical object of the present invention is to provide a polymer compound capable of binding to a cell membrane of NK cells.

Another technical object of the present invention is to provide a polymer compound capable of improving a surface modification efficiency of NK cells.

Another technical object of the present invention is to provide a polymer compound capable of recognizing a target cancer cell to a surface of NK cells.

Still another technical object of the present invention is to provide a polymer compound capable of selectively recognizing cancer cells including CD44.

Still another technical object of the present invention is to provide a polymer compound having an improved killing efficiency for cancer cells with CD44 overexpressed.

Still another technical object of the present invention is to provide a polymer compound capable of modifying a surface of NK cells without genetic manipulation.

Still another technical object of the present invention is to provide a polymer compound which may be naturally removed from NK cells without an external physical and chemical intervention.

The technical objects of the present invention are not limited to the above-described objects.

### Technical Solution

To solve the technical problems described above, the present invention may provide a polymer compound.

According to a first embodiment, there may be provided a polymer compound including a hydrophobic moiety binding to a natural killer (NK) cell, a cancer cell recognition moiety, and a linker, in which the hydrophobic moiety is bound to one end of the linker and the cancer cell recognition moiety is bound to the other end of the linker, so as to recognize NK cells and cancer cells, in which the cancer cell recognition moiety selectively recognizes solid cancer cells.

According to the first embodiment, the cancer cell recognition moiety may include hyaluronic acid.

According to the first embodiment, the polymer compound bound to NK cells through the hydrophobic moiety may be bound to cancer cells through the cancer cell recognition moiety, and then promote a secretion of cytotoxic granules and cytokines from NK cells to kill cancer cells.

According to the first embodiment, the hydrophobic moiety may include a lipid, and may be bound to a surface of the NK cell through a hydrophobic interaction via the lipid.

According to the first embodiment, the hydrophobic moiety may include any one of a phospholipid having an alkyl chain having 12 to 24 carbon atoms, a sterols lipid having 10 to 30 carbon atoms, 1,2-distearoyl-sn-glycero-3-phosphorylethanolamine (DSPE), 1,2-dioleoyl-sn-glycero-3-phosphoethanolamine (DPPE), and 1,2-dimyristoyl-sn-glycero-3-phosphoethanolamine (DMPE).

According to the first embodiment, the linker may prevent the polymer compound bound to the NK cell from being subjected to endocytosis into the NK cell.

According to the first embodiment, the linker may include polyethylene glycol (PEG).

According to the first embodiment, the polymer compound bound to the NK cell may be removed from the NK cell within 36 hours.

According to the first embodiment, the polymer compound bound to the NK cell may be naturally removed from the NK cell without an external physical and chemical intervention.

According to the first embodiment, the cancer cell recognition moiety may recognize pancreatic cancer cells (MIA PaCa-2), triple negative breast cancer cells (MDA-MB-231), and colorectal cancer cells (HCT-116).

According to the first embodiment, the polymer compound may inhibit the metastasis of solid cancer cells including CD44 to other organs.

According to a second embodiment, there may be provided a polymer compound including a hydrophobic moiety binding to a natural NK cell, a cancer cell recognition moiety, and a linker, in which the hydrophobic moiety is bound to one end of the linker and the cancer cell recognition moiety is bound to the other end of the linker, so as to recognize NK cells and cancer cells, in which the hydrophobic moiety includes a lipid for which a peak is not observed in the range of 6 to 9 ppm as a result of proton NMR analysis.

According to the second embodiment, the hydrophobic moiety may include 1,2-distearoyl-sn-glycero-3-phosphorylethanolamine (DSPE).

According to the second embodiment, the polymer compound may have a binding efficiency and a binding persistence for the NK cells improved as compared with a polymer compound including 1,2-dimyristoyl-sn-glycero-3-phosphoethanolamine (DMPE) as the hydrophobic moiety and a polymer compound including cholesterol as the hydrophobic moiety.

According to the second embodiment, the hydrophobic moiety may be bound to a surface of the NK cell through a hydrophobic interaction via the lipid.

According to the second embodiment, the polymer compound bound to the NK cell through the hydrophobic moiety may be bound to the cancer cell through the cancer cell recognition moiety, and then promote a secretion of cytotoxic granules and cytokines from the NK cell to kill the cancer cell.

According to the second embodiment, the cancer cell recognition moiety may selectively recognize solid cancer cells including CD44.

According to the second embodiment, the cancer cell recognition moiety may include hyaluronic acid.

According to the second embodiment, the linker may prevent the polymer compound bound to the NK cell from being subjected to endocytosis into the NK cell.

According to the second embodiment, the linker may include polyethylene glycol (PEG).

According to the second embodiment, the cancer cell recognition moiety may recognize pancreatic cancer cells (MIA PaCa-2), triple negative breast cancer cells (MDA-MB-231), and colorectal cancer cells (HCT-116).

According to the second embodiment, the polymer compound may inhibit the metastasis of solid cancer cells including CD44 to other organs.

According to a third embodiment, there may be provided a polymer compound including a hydrophobic moiety binding to a NK cell, a cancer cell recognition moiety, and a linker, in which the hydrophobic moiety is bound to one end of the linker and the cancer cell recognition moiety is bound to the other end of the linker, so as to recognize NK cells and cancer cells, in which a length of the linker and a content of the hydrophobic moiety are controlled to improve killing efficiency of solid cancer cells including CD44 through the NK cells.

According to the third embodiment, the linker may include polyethylene glycol (PEG).

According to the third embodiment, the linker may have a length greater than 0.6 k and less than 5 k.

According to the third embodiment, the hydrophobic moiety may include any one of a phospholipid having an alkyl chain having 12 to 24 carbon atoms, a sterols lipid having 10 to 30 carbon atoms, 1,2-distearoyl-sn-glycero-3-phosphorylethanolamine (DSPE), 1,2-dioleoyl-sn-glycero-3-phosphoethanolamine (DPPE), and 1,2-dimyristoyl-sn-glycero-3-phosphoethanolamine (DMPE).

According to the third embodiment, a degree of substitution (DS) defined as a value obtained by calculating an amount of the hydrophobic moiety linked to the cancer cell recognition moiety through an NMR analysis may be greater than 7% and less than 26%.

According to the third embodiment, the hydrophobic moiety may be bound to a surface of the NK cell through a hydrophobic interaction via a lipid.

According to the third embodiment, the cancer cell recognition moiety may include hyaluronic acid.

According to the third embodiment, the cancer cell recognition moiety may selectively recognize solid cancer cells including CD44.

According to the third embodiment, the polymer compound bound to the NK cell through the hydrophobic moiety may be bound to cancer cell through the cancer cell recognition moiety, and then promote a secretion of cytotoxic granules and cytokines from the NK cell to kill the cancer cell.

According to the third embodiment, the cancer cell recognition moiety may recognize pancreatic cancer cells (MIA PaCa-2), triple negative breast cancer cells (MDA-MB-231), and colorectal cancer cells (HCT-116).

According to the third embodiment, the polymer compound may inhibit the metastasis of solid cancer cells including CD44 to other organs.

To solve the technical problems described above, the present invention may provide a method for preparing a polymer compound.

According to a first embodiment, the method for preparing a polymer compound may include preparing hyaluronic acid, thiolating the hyaluronic acid, and reacting the thiolated hyaluronic acid with a compound in which a hydrophobic moiety is bound to one end of a linker and maleimide is bound to the other end thereof to prepare a polymer compound in which the hydrophobic moiety is bound to one end of the linker and the hyaluronic acid is bound to the other end thereof.

According to the first embodiment, the polymer compound may be prepared by a Michael reaction between the thiolated hyaluronic acid and a compound in which a hydrophobic moiety is bound to one end and maleimide is bound to the other end.

According to the first embodiment, the thiolating of the hyaluronic acid may include: conjugating the hyaluronic acid to 3-(2-pyridyldithithio)propionyl hydrazide (PDPH); and adding 2-mercaptoethanol to the hyaluronic acid to which the PDPH is conjugated.

According to a second embodiment, the method for preparing a polymer compound may include preparing hyaluronic acid, and subjecting the hyaluronic acid to a coupling reaction with a compound in which a hydrophobic moiety is bound to one end of a linker and an amine group is bound to the other end thereof to prepare a polymer compound in which the hydrophobic moiety is bound to one end of the linker and the hyaluronic acid is bound to the other end thereof.

According to the second embodiment, the preparing of the polymer compound may include adding and mixing the hyaluronic acid, 1-ethyl-3-(3-dimethylaminopropyl) carbodiimide (EDC), and N-hydroxy succinimide (NHS) in a solvent to prepare a base solution, and adding and stirring a compound in which a hydrophobic moiety is bound to one end and an amine group is bound to the other end in the base solution to perform an EDC/NHS coupling reaction between the hyaluronic acid and the compound.

According to the second embodiment, the hydrophobic moiety may include 1,2-distearoyl-sn-glycero-3-phosphorylethanolamine (DSPE).

According to the third embodiment, the method for preparing a polymer compound may include preparing hyaluronic acid, adding and mixing the hyaluronic acid, 1-ethyl-3-(3-dimethylaminopropyl) carbodiimide (EDC), and N-hydroxy succinimide (NHS) in a solvent to prepare a base solution, and adding and stirring a compound in which a hydrophobic moiety is bound to one end of a linker and an amine group is bound to the other end thereof in the base solution to prepare a polymer compound in which the hydrophobic moiety is bound to one end of the linker and the hyaluronic acid is bound to the other end thereof, in which a length of the linker and a content of the hydrophobic moiety in the polymer compound may be controlled to improve killing efficiency of solid cancer cells including CD44 through the NK cells.

According to the third embodiment, the linker may include polyethylene glycol (PEG) having a length greater than 0.6 k and less than 5 k.

According to the third embodiment, a degree of substitution (DS) defined as a value obtained by calculating an amount of the hydrophobic moiety linked to the cancer cell recognition moiety through an NMR analysis may be greater than 7% and less than 26%.

### Advantageous Effects

A polymer compound including a hydrophobic moiety (DSPE), a cancer cell recognition moiety (hyaluronic acid), and a linker (PEG) connecting the hydrophobic moiety and the cancer cell recognition moiety can easily modify a surface of NK cells by a hydrophobic interaction through the hydrophobic moiety (DSPE) without genetic manipulation (modification such that the NK cell can recognize a specific cancer cell), can selectively recognize solid cancer cells (e.g., pancreatic cancer cells, triple negative breast cancer cells, colon cancer cells, etc.) with CD44 overexpressed among various cancer cells, and can also have a validity evaluation for an animal model completed with respect to the pancreatic cancer cells.

In addition, the polymer compound can be naturally removed without an external physical and chemical intervention within a predetermined time (within 36 hours), and thus problems caused by a long-term surface modification of NK cells (e.g., cytokine release syndrome, neurotoxicity, off-tumor effects, acute respiratory distress syndrome, etc.) can be easily solved.

Furthermore, a killing efficiency of solid cancer cells including CD44 through the NK cells can be improved by controlling a length of the linker (more than 0.6 k and less than 5 k) and a content of the hydrophobic moiety (a degree of lipid substitution defined as a value obtained by calculating an amount of the hydrophobic moiety linked to the cancer cell recognition moiety through an NMR analysis is more than 7% and less than 26%).

### DESCRIPTION OF DRAWINGS

FIGS. 1 and 2 are views for describing a polymer compound according to an embodiment of the present invention and a state in which the polymer compound is bound to a natural killer (NK) cell.

FIG. 3 is a view for describing an endocytosis problem of a polymer compound bound to a NK cell.

FIG. 4 is a view for describing a cancer killing process through an NK cell to which a polymer compound is bound according to an embodiment of the present invention.

FIG. 5 is a view for describing a process in which a polymer compound is removed from an NK cell after being bound to the NK cell according to an embodiment of the present invention.

FIGS. 6 and 7 are views for describing a method for preparing a polymer compound according to an embodiment of the present invention.

FIG. 8 is a view comparing a HANK cell bound to AF-HA-SH and a HANK cell bound to AF-HA-PEG-Lipid.

FIGS. 9 and 10 are views for describing an effect of HA-PEG-Lipid on NK cells.

FIGS. 11 and 12 are views for confirming a target recognition ability of a HANK cell.

FIG. 13 is a graph comparing an amount of cytotoxic granules and cytokine secretion between NK cell and HANK cell for target cells.

FIG. 14 is a graph comparing a lysis ability between NK cell and HANK cell for cancer cells.

FIG. 15 and FIG. 16 are views comparing an anticancer efficacy between NK cell and HANK cell for tumor spheroids.

FIG. 17 is a view for confirming a retention time of HA-PEG-Lipid bound to NK cells.

FIG. 18 is a view comparing a cytokine secretion ability between NK cell and a restored NK cell.

FIG. 19 is a view comparing a cancer cell lysis ability between NK cell and a restored NK cell.

FIG. 20 is a view showing an experimental process for confirming an anticancer efficacy of HANK cell for pancreatic cancer.

FIGS. 21 and 22 are views showing a change in volume, weight, and size of a tumor according to a progress of an experiment on pancreatic cancer.

FIG. 23 is a view comparing a tumor penetration ability between NK cell and HANK cell.

FIG. 24 is a view for confirming a biodistribution of NK cell and HANK cell injected into experimental mice.

FIG. 25 and FIG. 26 are view comparing a cytotoxic granule spreading ability between NK cell and HANK cell.

FIG. 27 and FIG. 28 are views comparing a cytokine spreading ability between NK cell and HANK cell.

FIG. 29 and FIG. 30 are views comparing a tumor necrosis ability among PBS, NK cell, gemcitabine, and HANK cell.

FIG. 31 and FIG. 32 are views comparing an apoptotic region between tumor masses via PBS, NK cell, gemcitabine, and HANK cell.

FIG. 33 and FIG. 34 are view comparing a cell proliferation inhibitory ability among PBS, NK cell, gemcitabine, and HANK cell.

FIG. 35 is a view for describing a structural formula of a polymer compound according to Experimental Example 2 of the present invention.

FIG. 36 is a view for describing a result of proton NMR analysis on hyaluronic acid (HA).

FIG. 37 is a view for describing a result of proton NMR analysis on a HA-PEG-DMPE polymer compound.

FIG. 38 is a view for describing a result of proton NMR analysis on a HA-PEG-DSPE polymer compound.

FIG. 39 is a view for describing a result of proton NMR analysis on an HA-PEG-Chol polymer compound.

FIG. 40 is a view for confirming an endocytosis phenomenon of HADM-NK cell, HADS-NK cell, and HACH-NK cell.

FIG. 41 is a view for describing a coating efficacy of HADM-NK cell, HADS-NK cell, and HACH-NK cell.

FIG. 42 is a schematic view showing a flow cytometry for measuring a fluorescence intensity of HADM-NK cell, HADS-NK cell, and HACH-NK cell.

FIG. 43 is a view quantitatively showing a fluorescence intensity measured by the method of FIG. 42.

FIG. 44 is a view comparing an amount of cytokine secretion among NK cell, HADM-NK cell, HADS-NK cell, HACH-NK cell, and LPS-treated NK cell.

FIG. 45 is a view for comparing a content of TRAIL ligands and FasL ligands present on surfaces among NK cell, HADM-NK cell, HADS-NK cell, and HACH-NK cell.

FIG. 46 is a view for comparing the viability and cell proliferation ability among NK cell, HADM-NK cell, HADS-NK cell, and HACH-NK cell.

FIGS. 47 and 48 are views for describing a target recognition ability and anticancer efficacy of NK cell, HADM-NK cell, HADS-NK cell, and HACH-NK cell.

FIGS. 49 and 50 are views showing a result of FT-IR analysis on polymer compounds according to experimental examples of the present invention.

FIG. 51 is a view showing a result of hydrophobic analysis on polymer compounds according to experimental examples of the present invention.

FIG. 52 is an image of measured fluorescence intensity and a graph of measured fluorescence intensity with regard to cells in which the polymer compounds are bound to NK cells according to experimental examples of the present invention.

FIG. 53 is a graph showing a quantitative comparison of NK cell surface modification efficiency among polymer compounds according to experimental examples of the present invention.

FIG. 54 is a graph summarizing an NK cell surface modification efficiency of the polymer compounds with respect to a log P value according to experimental examples of the present invention.

FIG. 55 is a graph showing a coating sustainability and cell proliferation ability of cells in which polymer compounds are bound to NK cells according to experimental examples of the present invention.

FIG. 56 is a graph for describing an effect of polymer compounds on a ligand of NK cell according to experimental examples of the present invention.

FIG. 57 is a graph for describing an effect of polymer compounds on a cytokine secretion of NK cell according to experimental examples of the present invention.

FIG. 58 is a view for confirming a targeting ability of NK cell, to which polymer compounds are bound, for triple negative breast cancer cells according to experimental examples of the present invention.

FIG. 59 is a graph quantifying an E:T cluster ratio measured in FIG. 58.

FIG. 60 is a view for confirming a targeting ability of NK cell, to which polymer compounds are bound, for liver cancer cells according to experimental examples of the present invention.

FIG. 61 is a graph quantifying an E:T cluster ratio measured in FIG. 60.

FIG. 62 is a view for confirming a targeting ability of NK cell, to which polymer compounds are bound, for fibroblasts according to experimental examples of the present invention.

FIG. 63 is a graph quantifying an E:T cluster ratio measured in FIG. 62.

FIG. 64 is a view for describing a killing ability of NK cell, to which polymer compounds are bound, for triple negative breast cancer cells according to experimental examples of the present invention.

FIG. 65 is a view for describing a killing ability of NK cell, to which polymer compounds are bound, for liver cancer cells according to experimental examples of the present invention.

FIG. 66 is a view for describing a killing ability of NK cell, to which polymer compounds are bound, for fibroblasts according to experimental examples of the present invention.

### MODE FOR INVENTION

Hereinafter, preferred embodiments of the present invention will be described in detail with reference to the accompanying drawings. However, the technical spirit of the present invention is not limited to the embodiments described herein and may be implemented in other forms. Rather, the embodiments introduced herein are provided so that the disclosed contents may be thorough and complete and the spirit of the present invention may be sufficiently conveyed to those skilled in the art.

In this specification, when a component is referred to as being on another component, it means that it may be formed directly on the other component or a third component may be interposed therebetween. In addition, in the drawings, the thicknesses of films and regions are exaggerated for effective description of the technical contents.

Furthermore, in various embodiments of the present specification, terms such as first, second, third, etc., are used to describe various components, but these components should not be limited by these terms. These terms have only been used to distinguish one component from another component. Accordingly, a component mentioned as a first component in one embodiment may be mentioned as a second component in another embodiment. Each embodiment described and exemplified herein includes a complementary embodiment thereof. In addition, in the present specification, “and/or” is used as a meaning including at least one of the components listed before and after.

In the specification, a singular expression includes a plural expression unless the context clearly indicates otherwise. In addition, terms such as “include,” “have” or the like are intended to designate the presence of features, numbers, steps, components, or combinations thereof described in the specification, and should not be understood to preclude the possibility of the presence or addition of one or more other features, numbers, steps, components, or combinations thereof. In addition, in the present specification, “connection” is used as a meaning including both indirectly connecting a plurality of components and directly connecting the plurality of components.

Furthermore, in the following description of the present invention, when it is determined that a detailed description of a related known function or configuration may unnecessarily obscure the subject matter of the present invention, the detailed description thereof will be omitted.

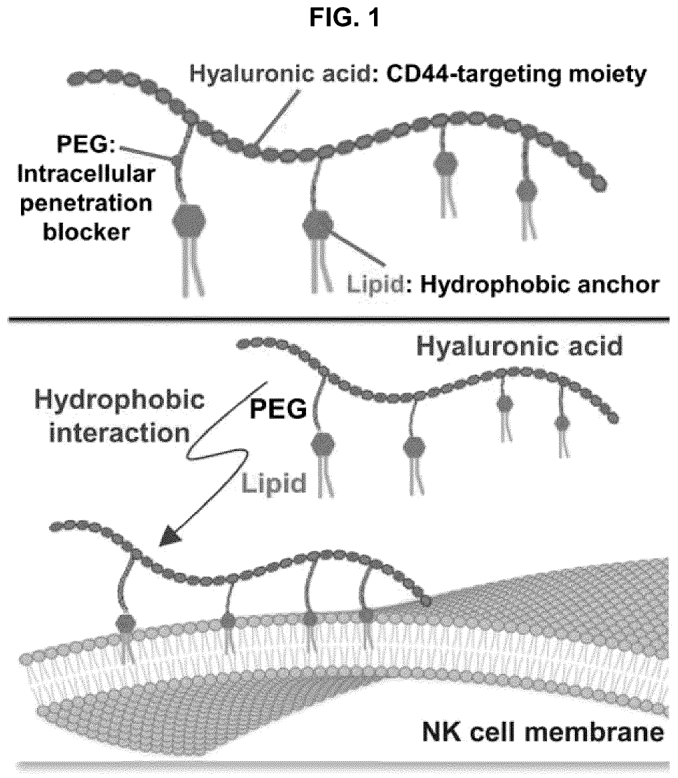

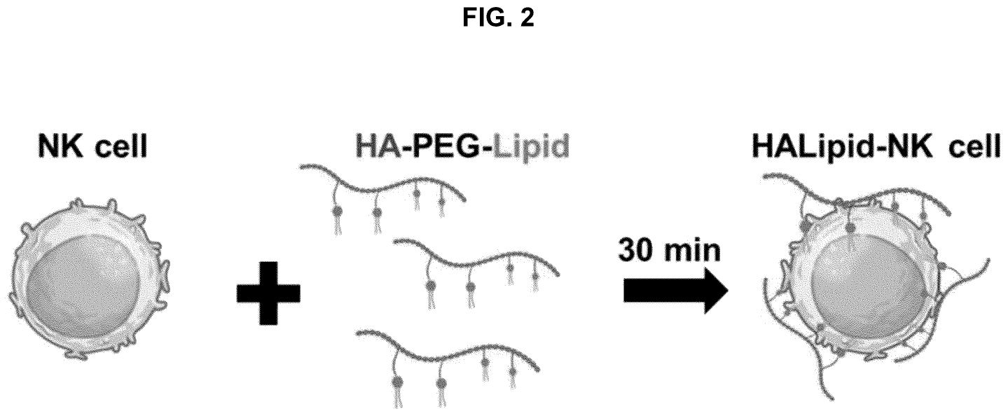

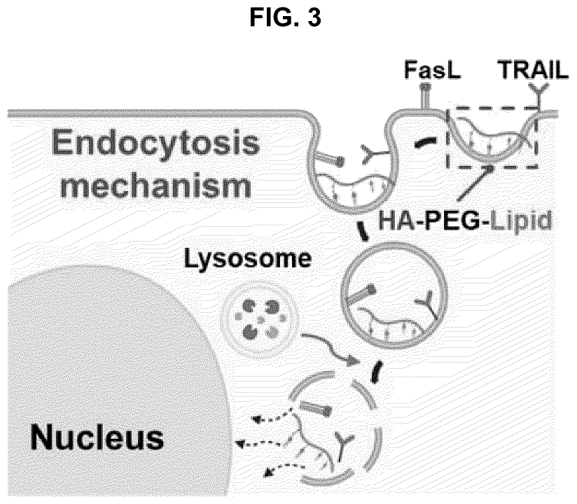

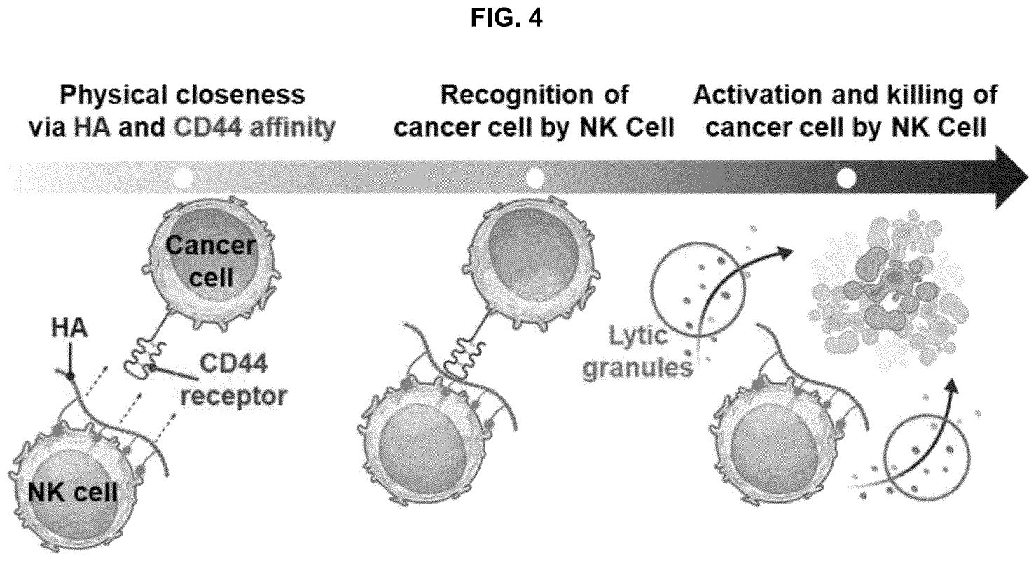

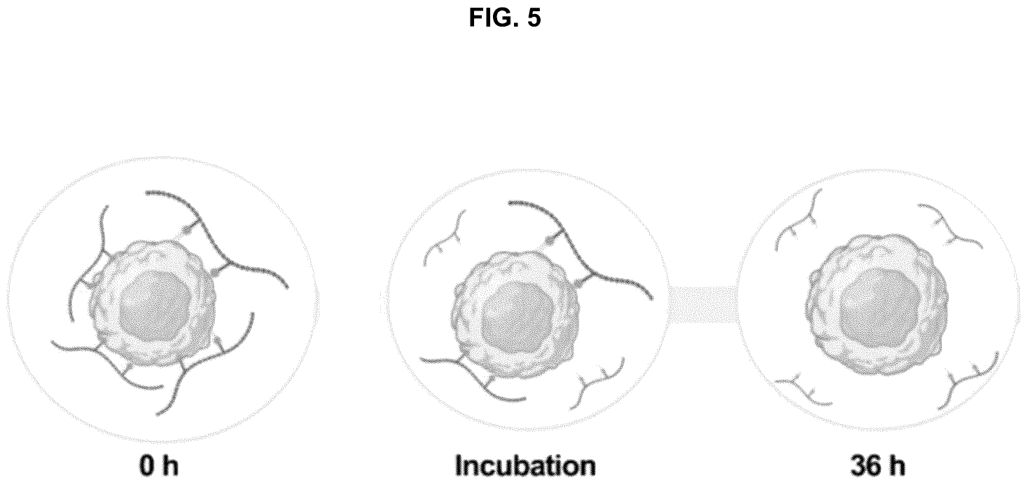

FIGS. 1 and 2 are views for describing a polymer compound according to an embodiment of the present invention and a state in which the polymer compound is bound to a natural killer (NK) cell, FIG. 3 is a view for describing an endocytosis problem of a polymer compound bound to a NK cell, FIG. 4 is a view for describing a cancer killing process through an NK cell to which a polymer compound is bound according to an embodiment of the present invention, and FIG. 5 is a view for describing a process in which a polymer compound is removed from an NK cell after being bound to the NK cell according to an embodiment of the present invention.

Referring to FIGS. 1 and 2, the polymer compound according to an embodiment of the present invention may include a hydrophobic moiety, a cancer cell recognition moiety, and a linker connecting the hydrophobic moiety and the cancer cell recognition moiety. In other words, the polymer compound may have a structure in which the hydrophobic moiety is bound to one end of the linker and the cancer cell recognition moiety is bound to the other end thereof.

The hydrophobic moiety may recognize a natural killer (NK) cell and may be bound to a surface of the NK cell. Accordingly, the polymer compound may be immobilized on the surface of the NK cell by the hydrophobic moiety. More specifically, the hydrophobic moiety may be configured to include a lipid, and may be bound to the surface of the NK cell through a hydrophobic interaction via the lipid.

Examples of a material which may be used as the hydrophobic moiety may include a phospholipid having an alkyl chain having 12 to 24 carbon atoms, a sterols lipid having 10 to 30 carbon atoms, 1,2-distearoyl-sn-glycero-3-phosphorylethanolamine (DSPE), 1,2-dioleoyl-sn-glycero-3-phosphoethanolamine (DPPE), 1,2-dimyristoyl-sn-glycero-3-phosphoethanolamine (DMPE), and the like.

However, in the case of other lipids except 1,2-distearoyl-sn-glycero-3-phosphorylethanolamine (DSPE), there may be a problem in that the binding efficiency and binding persistence with NK cells are low. For example, 1,2-dimyristoyl-sn-glycero-3-phosphoethanolamine (DMPE) may have a problem in that the binding efficiency to NK cells is significantly low. Specifically, when the polymeric compound is bound to the surface of the NK cell through DMPE, as shown in FIG. 3, there may be a problem of endocytosis in which the polymeric compound (HA-PEG-Lipid) is introduced into the NK cell. In addition, when the polymer compound is bound to the surface of the NK cell through cholesterol, there may be a problem in that the binding efficiency is good, but the polymer compound is removed from the NK cell in a short time.

However, when 1,2-distearoyl-sn-glycero-3-phosphorylethanolamine (DSPE) is used as the hydrophobic moiety, not only the binding efficiency but also the binding persistence may be high, so that the cancer cell killing efficiency through NK cells may be significantly improved.

The cancer cell recognition moiety may recognize a cancer cell and bind to the cancer cell, and may include hyaluronic acid. As described above, when the cancer cell recognition moiety includes hyaluronic acid, the cancer cell recognition moiety may selectively recognize solid cancer cells including CD44. More specifically, the cancer cell recognition moiety including hyaluronic acid may selectively recognize cancer cells with CD44 overexpressed. For example, cancer cells with CD44 overexpressed may include pancreatic cancer cells (MIA PaCa-2), triple negative breast cancer cells (MDA-MB-231), and colon cancer cells (HCT-116). In other words, the cancer cell recognition moiety including hyaluronic acid may selectively recognize pancreatic cancer cells (MIA PaCa-2), triple negative breast cancer cells (MDA-MB-231), and colorectal cancer cells (HCT-116) among various cancer cells. Accordingly, the NK cell bound to the hydrophobic moiety may selectively kill pancreatic cancer cells (MIA PaCa-2), tritium breast cancer cells (MDA-MB-231), and colorectal cancer cells (HCT-116) among various cancer cells by the cancer cell recognition moiety.

The linker may be adapted for connecting the hydrophobic moiety and the cancer cell recognition moiety, and may include polyethylene glycol (PEG). In addition, the linker may prevent the polymer compound bound to the NK cell from being subjected to endocytosis into the NK cell.

Referring to FIG. 4, a process in which the polymer compound bound to the NK cell through the hydrophobic moiety kills cancer cells may be shown.

Specifically, the NK killer cell bound to the polymer compound may recognize cancer cells including CD44 through the cancer cell recognition moiety (hyaluronic acid, HA) of the polymer compound, and then may be bound to cancer cells through the cancer cell recognition moiety (hyaluronic acid, HA). After that, an activation process in which cytotoxic granules and cytokines are secreted from the NK cell is performed, and cancer cells may be killed by the cytotoxic granules and cytokines secreted from the NK cell. According to one embodiment, the polymer compound may promote the secretion of cytotoxic granules and cytokines from the NK cell during the activation process, thereby improving cancer cell killing efficiency.

Referring to FIG. 5, the polymer compound bound to the NK cells may be naturally removed from the NK cell within 36 hours without an external physical and chemical intervention. Unlike the above description, when the polymer compound bound to the NK cell is not removed, various problems such as cytokine release syndrome, neurotoxicity, off-tumor effects, and acute respiratory distress syndrome may occur.

In order to solve these problems, a method of introducing and removing a suicide gene has been conventionally used, but another problem has occurred because additional drug treatment for activating the suicide gene needs to be performed. However, since the polymer compound may be naturally removed from the NK cell without an external physical and chemical intervention, the above-described problems may be easily solved.

In addition, the binding and removal of the polymer compound for the NK cell may not impair an intrinsic function of the NK cells. In other words, even after the polymer compound is removed from the NK cell, the NK cell may maintain an inherent functions thereof.

According to one embodiment, the polymer compound may control a length of the linker (PEG) to improve the killing efficiency of solid cancer cells including CD44 through the NK cell. More specifically, the linker (PEG) may have a length greater than 0.6 k and less than 5 k. On the contrary, when the length of the linker (PEG) is 0.6 k or less or 5 k or more, a surface modification efficiency of the NK cell through the polymer compound (a content of the polymer compound bound to the NK cell) may be significantly reduced. In other words, when the length of the linker (PEG) is 0.6 k or less or 5 k or more, there may occur a problem in that the polymer compound is not bound to the surface of the NK cell. Accordingly, the cancer cell killing efficiency using the NK cell bound to the polymer compound may be significantly reduced.

In addition, according to one embodiment, the polymer compound may control the content of the hydrophobic moiety (e.g., DSPE) to improve the killing efficiency of solid cancer cells including CD44 through the NK cell. More specifically, the polymer compound may have a degree of substitution (DS) of more than 7% and less than 26%, which is defined as a value obtained by calculating an amount of the hydrophobic moiety (DSPE) linked to the cancer cell recognition moiety (hyaluronic acid) through an NMR analysis. On the contrary, when the DS is 7% or less or 26% or more, the surface modification efficiency of the NK cell through the polymer compound (the content of the polymer compound bound to the NK cell) may be significantly reduced. In other words, when the DS is 7% or less or 26% or more, there may occur a problem in that the polymer compound is not bound to the surface of the NK cell. Accordingly, the cancer cell killing efficiency using the NK cell bound to the polymer compound may be significantly reduced.

As a result, a polymer compound including a hydrophobic moiety (DSPE), a cancer cell recognition moiety (hyaluronic acid), and a linker (PEG) connecting the hydrophobic moiety and the cancer cell recognition moiety may easily modify a surface of NK cells by a hydrophobic interaction through the hydrophobic moiety (DSPE) without genetic manipulation (modification such that the NK cell may recognize a specific cancer cell), may selectively recognize solid cancer cells (e.g., pancreatic cancer cells, triple negative breast cancer cells, colon cancer cells, etc.) with CD44 overexpressed among various cancer cells, and may also have a validity evaluation for an animal model completed with respect to the pancreatic cancer cells.

In addition, the polymer compound may be naturally removed without an external physical and chemical intervention within a predetermined time (within 36 hours), and thus problems caused by a long-term surface modification of NK cells (e.g., cytokine release syndrome, neurotoxicity, off-tumor effects, acute respiratory distress syndrome, etc.) may be easily solved.

Furthermore, the killing efficiency of solid cancer cells including CD44 through the NK cells may be improved by controlling a length of the linker (more than 0.6 k and less than 5 k) and a content of the hydrophobic moiety (a degree of lipid substitution defined as a value obtained by calculating an amount of the hydrophobic moiety linked to the cancer cell recognition moiety through an NMR analysis is more than 7% and less than 26%).

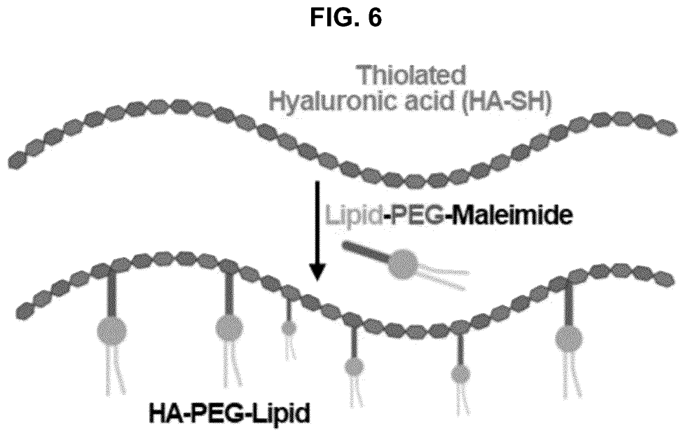

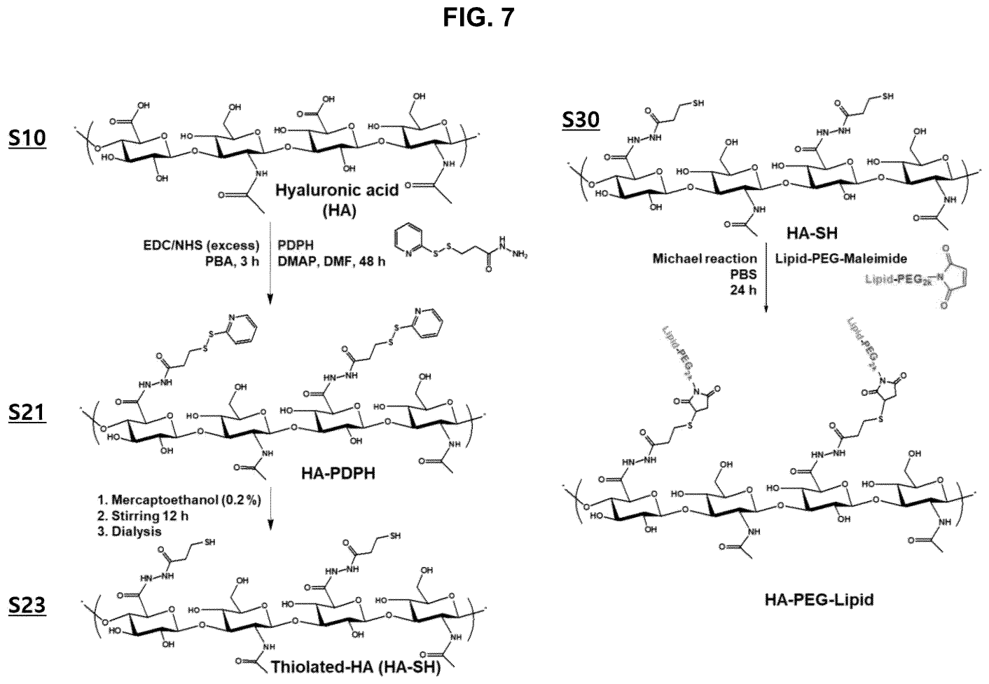

FIGS. 6 and 7 are views for describing a method for preparing a polymer compound according to an embodiment of the present invention.

Referring to FIGS. 6 and 7, the method for preparing a polymer compound according to an embodiment of the present invention may include preparing hyaluronic acid (S10), thiolating the hyaluronic acid (S21, S22), and preparing the polymer compound by using the thiolated hyaluronic acid. Hereinafter, each step will be described in detail.

In above S10, hyaluronic acid (HA) may be prepared. According to one embodiment, in above S10, 1-ethyl-3-(3-dimethylaminopropyl) carbodiimide (EDC) and N-hydroxy succinimide (NHS) may be added to a mixed solution of a hyaluronic acid solution and phosphate-buffered saline (PBS) to activate a carboxyl group.

In above S21, the hyaluronic acid may be conjugated with 3-(2-pyridyldithiothio)propionyl hydrazide (PDPH). According to one embodiment, in above S21, the hyaluronic acid prepared in above S10 may be mixed with 3-(2-pyridyldithithio)propionyl hydrazide (PDPH), 4-dimethyl amino pyridine (DMAP), and dimethyl formamide (DMF) to conjugate the hyaluronic acid with the PDPH. The compound in which the hyaluronic acid is conjugated with PDPH may be defined as HA-PDPH.

In above S22, 2-mercaptoethanol may be added to the hyaluronic acid with which the PDPH is conjugated (HA-PDPH) to prepare a thiolated hyaluronic acid (thiolated-HA, HA-SA).

Finally, in above S30, the thiolated hyaluronic acid (HA-SH) may be subjected to a Michael reaction with a compound in which a hydrophobic moiety (lipid) is bound to one end of the linker and maleimide is bound to the other end thereof. Accordingly, the polymer compound (HA-PEG-Lipid) in which the hydrophobic moiety (lipid) is bound to one end of the linker (PEG) and the hyaluronic acid (HA) is bound to the other end thereof may be prepared.

Hereinafter, the polymer compound according to an embodiment of the present invention will be described in more detail through specific experimental examples.

### Preparation of Polymer Compound (HA-PEG-Lipid) According to Experimental Example 1

A hyaluronic acid (HA) solution (Mw 60 k, 10 mg/mL, 1 equivalent, LifeCore Biomedical) and phosphate-buffered saline (PBS, pH 7.4) were stirred for 30 minutes, after which an excessive amount of 1-ethyl-3-(3-dimethylaminopropyl) carbodiimide (EDC, Sigma-Aldrich) and N-hydroxy succinimide (NHS, Sigma-Aldrich) were added to the stirred solution and reacted at room temperature for three hours to activate a carboxyl group.

After that, 3-(2-pyridyldithione)propionyl hydrazide (PDPH, Sigma-Aldrich) (30 equivalents), 2 mg of 4-dimethyl amino pyridine (DMAP, Sigma-Aldrich), and dimethyl formamide (DMF, Sigma-Aldrich) were further added thereto and reacted at room temperature for 72 hours to obtain a HA-PDPH product in which PDPH was conjugated to HA. In addition, the resulting HA-PDPH product was dialyzed (MWCO 2 kDa) with distilled water for three days and lyophilized to remove unconjugated PDPH and EDC/NHS therefrom.

After 30 mg of HA-PDPH was dissolved in 10 mL of PBS, 2-mercaptoethanol was added at an initial concentration of 0.2 wt % and stirred at room temperature for 12 hours. A mixture resulting from stirring was dialyzed (MWCO 2 kDa) with distilled water for three days and then lyophilized to prepare a thiolated HA (HA-SH).

50 mg of HA-SH was dissolved in 10 mL of PBS to prepare a homogeneous HA-SH solution, and then a solution in which Lipid-PEG-Maleimide (30 eq.) was dissolved in 10 mL of DMF was added to the resulting solution, and stirred at room temperature for 24 hours. The stirred mixture was dialyzed (MWCO 12-14 kD) with distilled water for three days and lyophilized to prepare HA-PEG-Lipid as a final product. More specifically, 1,2-distearoyl-sn-glycero-3-phosphorylethanolamine (DSPE) was used as the lipid.

### Preparation of HANK Cell According to Experimental Example 1

The polymer compound (HA-PEG-Lipid) according to the experimental example was dissolved in MEM alpha, and 5×10⁵ NK-92 mi NK cells were evenly mixed in 100 μL of a solution, and then reacted at room temperature for 30 minutes to prepare HANK cells in which the polymer compound (HA-PEG-Lipid) according to the experimental example was bound to NK cells.

### Experimental Example 1-1: Analysis of HA-PEG-Lipid and HANK Cell Properties

20 mg of HA-PEG-Lipid was dissolved in 2 mL of PBS and 100 nmol of Alex Flour 488 hydrazide (fluorescent dye) was added to prepare AF-HA-PEG-Lipid. In addition, 20 mg of HA-SH was dissolved in 2 mL of PBS and 100 nmol of Alex Flour 488 hydrazide (fluorescent dye) was added to prepare AF-HA-SH. After that, AF-HA-PEG-Lipid and AF-HA-SH were bound to NK cells to prepare HANK cells.

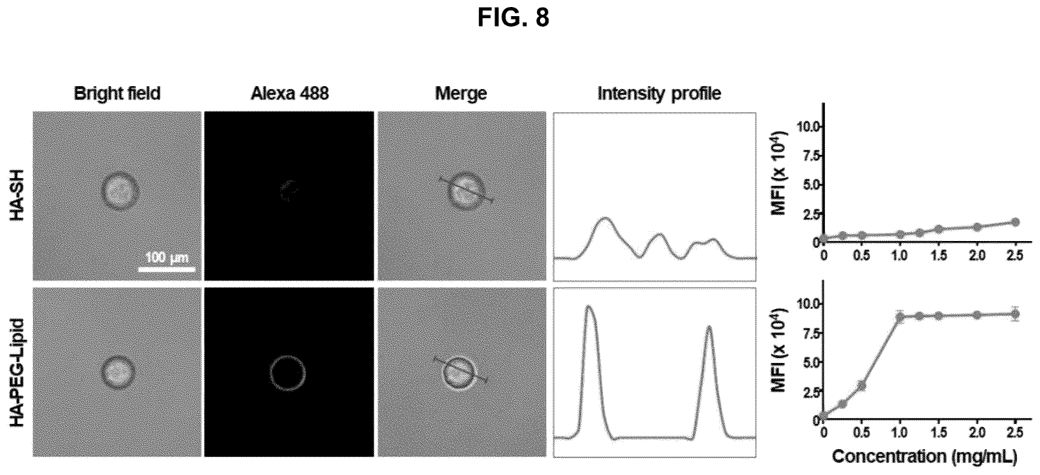

FIG. 8 is a view comparing a HANK cell bound to AF-HA-SH and a HANK cell bound to AF-HA-PEG-lipid.

Referring to FIG. 8, it shows the optical and fluorescence microscopy images of HANK cell bound to AF-HA-SH and HANK cell bound to AF-HA-PEG-Lipid, respectively, a result of intensity profile analysis on green fluorescence at a red line of the microscopy images, and a mean fluorescence intensity (MFI, ×10⁴) detected by flow cytometry.

As can be seen in the fluorescence microscope image of FIG. 8, it can be confirmed that AF-HA-SH is internalized into NK cells, whereas AF-HA-PEG-Lipid is uniformly bound to the surface of the NK cells. In addition, as can be seen in the mean fluorescence intensity detected by flow cytometry, it could be confirmed that in the case of HANK cell bound to AF-HA-PEG-Lipid, an MFI value is saturated at a concentration of 1 mg/mL, whereas in the case of HANK cell bound to AF-HA-SH, a low MFI of 2.5 or less is exhibited even at a concentration of 2.5 mg/mL. In other words, it could be confirmed that in the case of HA-SH, there is no moiety recognizing the NK cell, and thus it was not immobilized to the surface of the NK cell, whereas in the case of HA-PEG-Lipid, it is easily bound to the surface of the NK killer cell by lipid.

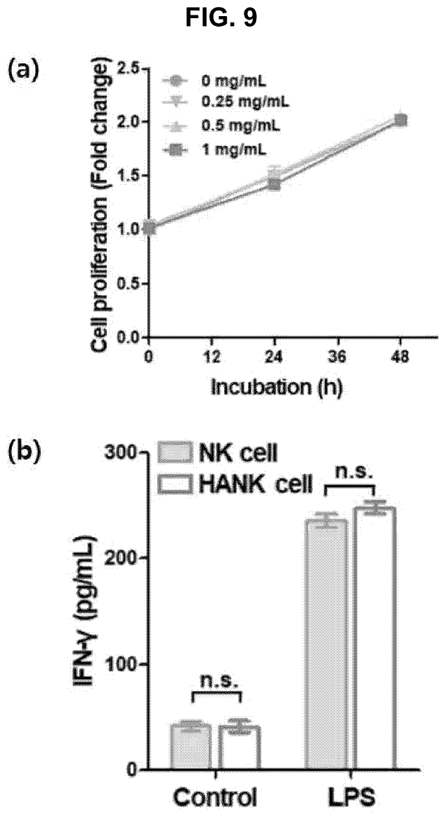

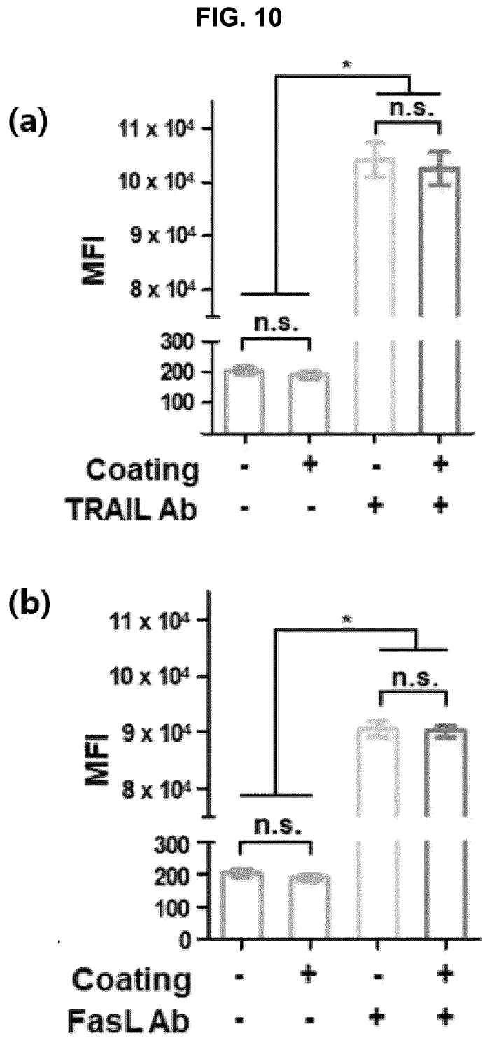

FIGS. 9 and 10 are views for describing an effect of HA-PEG-Lipid on NK cells.

Referring to (a) of FIG. 9, the survival rate and proliferation rate of HANK cells in which 0 to 1 mg/mL of HA-PEG-Lipid is bound to NK cells are shown. As can be seen in (a) of FIG. 9, it can be confirmed that the survival rate and the proliferation rate are maintained in a substantially constant way despite the binding of HA-PEG-Lipid to NK cells.

Referring to (b) of FIG. 9, the result of applying LPS to HANK cell as a proinflammatory antigen signal is shown. LPS may bind to TLR4 of immune cells and induce a secretion of inflammatory cytokines such as IFN-Y. As can be seen in (b) of FIG. 9, it could be confirmed that both NK cell and HANK cell treated with LPS exhibit a similar level of IFN-Y secretion. In other words, it can be seen from (a) and (b) of FIG. 9 that HA-PEG-Lipid bound to the surface of the NK cell does not interfere with antigen recognition and subsequent cytokine secretion processes after intracellular signal transduction.

(a) of FIG. 10 may show the results of MFI analysis by flow cytometry after detecting TRAIL present on the surface of HANK cell using APC-bound TRAIL antibody, and (b) of FIG may show the results of making an MFI analysis by flow cytometry after detecting FasL present on the surface of HANK cell using APC-bound FasL antibody.

FasL and TRAIL may be major surface ligands of NK cells which may recognize target cancer cells and induce direct killing, and as can be seen in (a) and (b) of FIG. 10, it could be confirmed that HANK cell exhibits an MFI similar to that of antibody-treated NK cell. In other words, it can be seen that HA-PEG-Lipid does not interfere with the availability of ligands on the surface of NK cells.

### Experimental Example 1-2: In Vitro Anticancer Effect Mediated by Immune Synapse

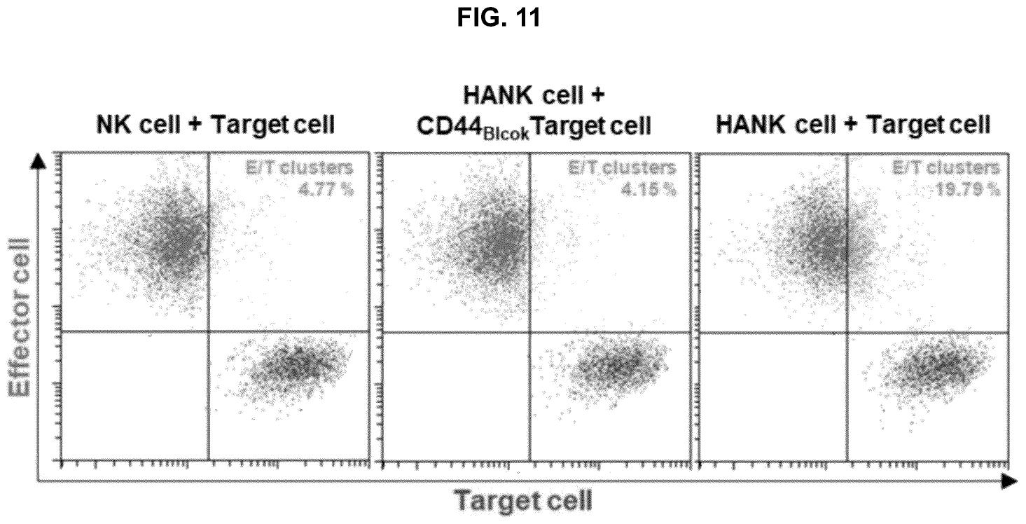

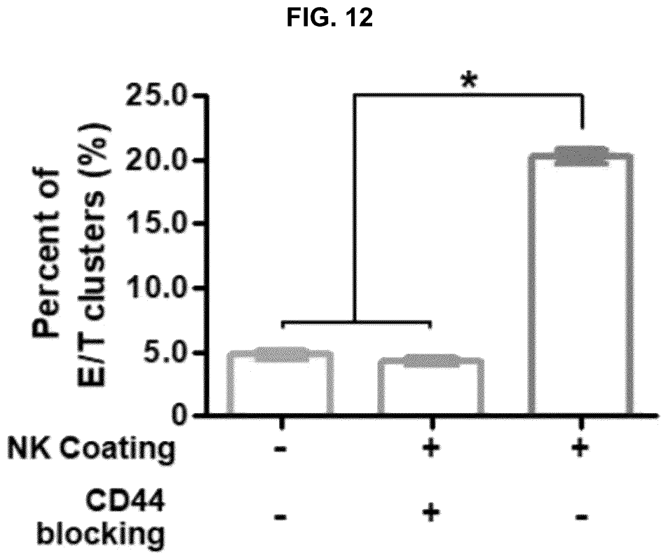

FIGS. 11 and 12 are views for confirming a target recognition ability of a HANK cell.

Referring to FIGS. 11 and 12, the target recognition ability of NK cell for target cell (NK cell+Target cell), the target recognition ability of HANK cell for CD44_{Block} target cell (HANK cell+CD44_{Block} Target cell), and the target recognition ability of HANK cell for target cell (HANK cell+Target cell) are measured and shown. Specifically, CD44 positive cancer cell lines (MIA PaCa-2, MDA-MB-231, and HCT-116) were used as a target cell, and cells in which the above-described target cell was pre-treated with 1 mg/mL of hyaluronic acid (HA) for two hours to block CD44 were used as the CD44_{Block} target cell. In addition, the target recognition ability was assessed by quantification of effector/target (E/T) cluster.

As can be seen in FIGS. 11 and 12, it could be confirmed that the target recognition ability of NK cell for target cell (NK cell+Target cell) and the target recognition ability of HANK cell for CD44_{Block} Target cell (HANK cell+CD44_{Block} Target cell) are 4.77% and 4.15%, respectively, whereas the target recognition ability of HANK cell for target cell (HANK cell+Target cell) is 19.79%, which is significantly high. In other words, it can be seen that HANK cell has a high target recognition ability for CD44.

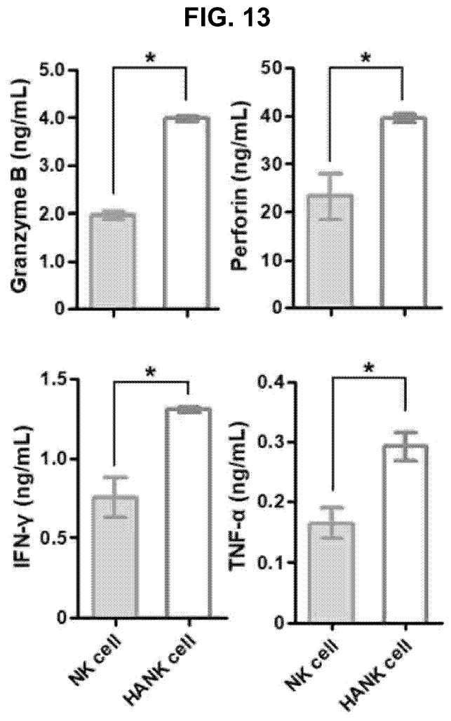

FIG. 13 is a graph comparing an amount of cytotoxic granules and cytokine secretion between NK cell and HANK cell for target cells.

Referring to FIG. 13, after co-incubating the target cell and the NK cell, an amount of secretion of cytotoxic granules (Granzyme B, Perforin) and cytokines (IFN-Y, TNF-α) released from the NK cell are measured and shown. In addition, after co-incubating the target cell and the HANK cell, an amount of secretion of cytotoxic granules (Granzyme B, Perforin) and cytokines (IFN-Y, TNF-α) released from the HANK cell are measured and shown. MIA PaCa-2 was used as the target cell.

As can be seen in FIG. 13, it can be confirmed that HANCK cell leads to an active secretion of cytotoxic granules (Granzyme B, Perforin) and cytokines (IFN-Y, TNF-α) compared to NK cell. In other words, it can be seen that the secretion of cytotoxic granules (Granzyme B, Perforin) and cytokines (IFN-Y, TNF-α) from NK cell is promoted by HA-PEG-Lipid.

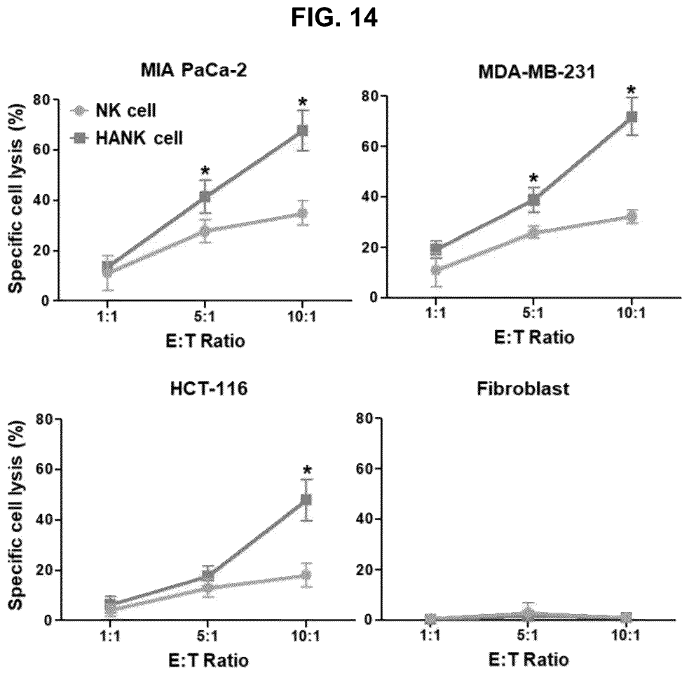

FIG. 14 is a graph comparing a lysis ability between NK cell and HANK cell for cancer cells.

Referring to FIG. 14, the NK cell and HANK cell are incubated with cancer cells (MIA PaCa-2, MDA-MB-231, HCT-116, and fibroblast), respectively, and then a cancer cell lysis rate (specific cell lysis, %) is measured and shown. An E:T ratio shown in FIG. 14 may mean an incubation ratio of NK cell or HANK cell:cancer cell.

As can be seen in FIG. 14, it could be confirmed that HANK cell exhibits a significantly higher lysis rate compared to NK cell for MIA PaCa-2, MDA-MB-231, and HCT-116 with CD44 overexpressed. In other words, it can be seen that the target recognition ability of NK cell for MIA PaCa-2, MDA-MB-231, and HCT-116 is improved by HA-PEG-Lipid. In addition, it could be confirmed that HANK cell fails to lyse fibroblasts without CD44. In other words, it can be seen that HANK cell selectively recognizes cancer cells with CD44 overexpressed among various cancer cells.

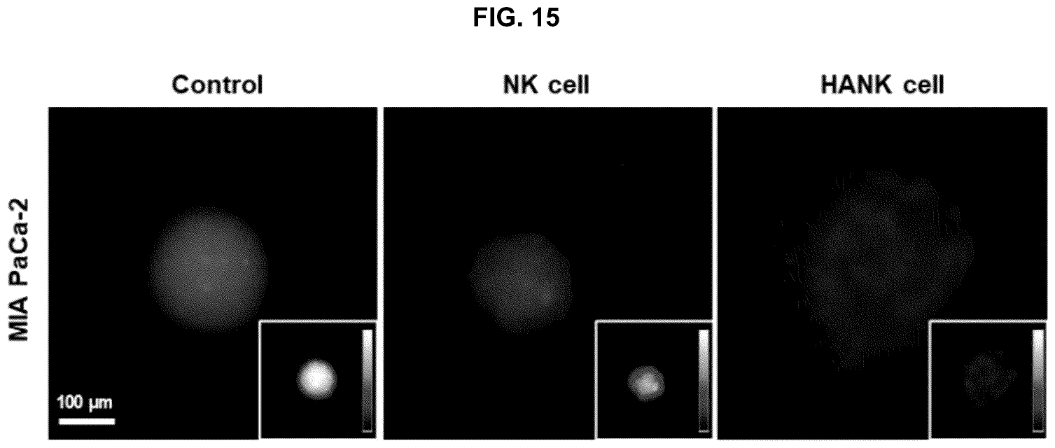

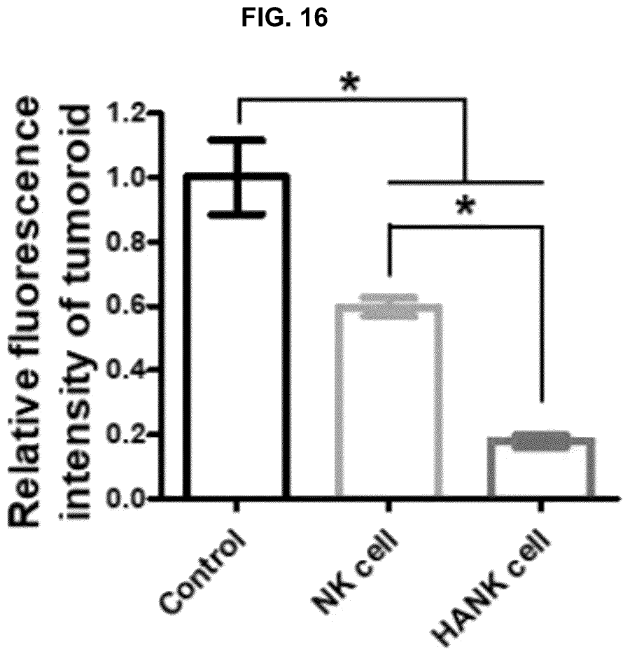

FIG. 15 and FIG. 16 are views comparing an anticancer efficacy between NK cell and HANK cell for tumor spheroids.

Referring to FIGS. 15 and 16, tumor spheroids were incubated with NK cell and HANK cell, respectively, and then an anticancer efficacy was measured by measuring a fluorescence intensity. A control shown in FIGS. 15 and 16 may mean a tumor spheroid.

As can be seen in FIG. 15, it can be confirmed that a morphology of tumor spheroids incubated with HANK cell is significantly destroyed, and as can be seen in FIG. 16, it can be confirmed that a fluorescence intensity of tumor spheroids incubated with HANK cell is significantly reduced compared to tumor spheroids incubated with NK cell.

### Experimental Example 1-3: Restoration of HANK Cell to NK Cell

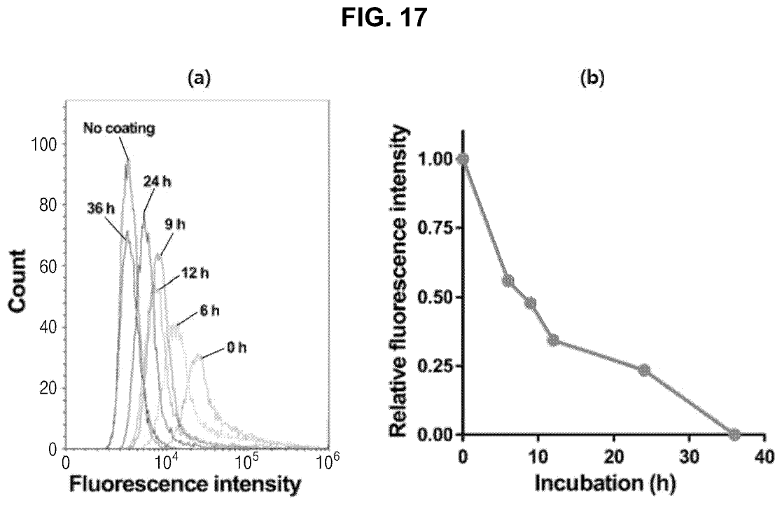

FIG. 17 is a view for confirming a retention time of HA-PEG-Lipid bound to NK cells.

Referring to FIG. 17, a retention time of HA-PEG-Lipid bound to NK cell is confirmed and shown through a fluorescence intensity analysis. As can be seen in FIG. 17, it can be confirmed that HA-PEG-Lipid bound to NK cell is removed from NK cell within 36 hours without an external physical and chemical intervention.

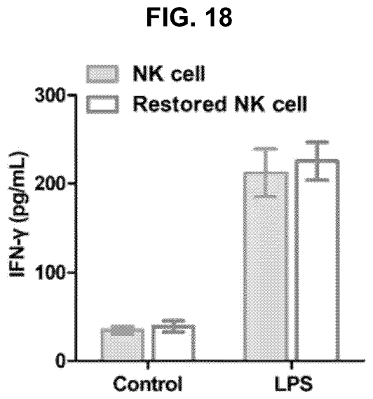

FIG. 18 is a view comparing a cytokine secretion ability between NK cell and a restored NK cell.

Referring to FIG. 18, the abilities to secrete cytokines (IFN-Y, pg/mL) for each of NK cell and the restored NK cell are shown in comparison with each other. The restored NK cell may mean a state in which the HA-PEG-Lipid is bound to NK cell to form the HANK cell and then the HA-PEG-Lipid is removed from the NK cell. In addition, the control of FIG. 18 may show a general state, and LPS may show the result of applying LPS as an inflammatory antigen signal. LPS may bind to TLR4 of immune cells and induce a secretion of inflammatory cytokines such as IFN-Y.

As can be seen in FIG. 18, it could be confirmed that there is no substantial difference between the NK cell and the restored NK cell with regard to the cytokine secretion ability. In other words, it can be seen that the binding and removal of HA-PEG-Lipid does not affect the cytokine secretion ability of NK cell.

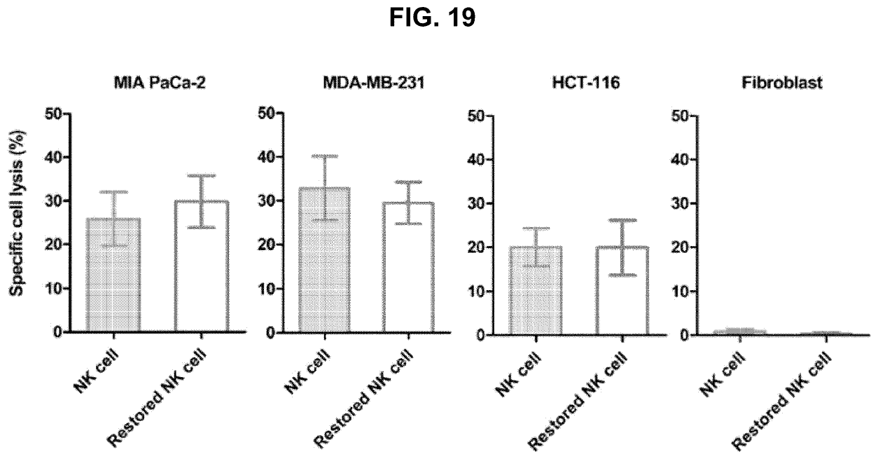

FIG. 19 is a view comparing the cancer cell lysis abilities between NK cell and a restored NK cell.

Referring to FIG. 19, the abilities to lyse cancer cells for each of NK cell and restored NK cell are shown in comparison with each other MIA PaCa-2, MDA-MB-231, and HCT-116 with CD44 overexpressed and fibroblast without CD44 were used as cancer cells.

As can be seen in FIG. 19, it could be confirmed that there is no substantial difference between the NK cell and the restored NK cell with regard to the ability to lyse MIA PaCa-2, MDA-MB-231, HCT-116 cancer cells. In other words, it can be seen that the binding and removal of HA-PEG-Lipid does not affect the ability of NK cell to lyse cancer cells. In addition, it can be seen that the restored NK cell still does not have the ability to lyse fibroblast without CD44.

### Experimental Example 1-4: Evaluation of Anticancer Efficacy of HANK Cell for Pancreatic Cancer

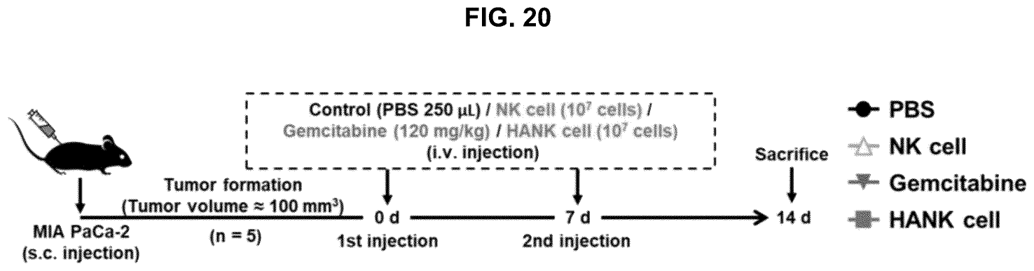

FIG. 20 is a view showing an experimental process for confirming an anticancer efficacy of HANK cell for pancreatic cancer.

Referring to FIG. 20, MIA PaCa-2, which is a pancreatic cancer cell, was injected into an experimental mouse to form a tumor, and then PBS (250 μL), NK cell (10⁷ cell), gemcitabine (120 mg/kg), and HANK cell (10⁷ cell) were administered thereto to evaluate an anticancer efficacy thereof, respectively. PBS was used as a control, and gemcitabine was used to confirm the efficacy of HANK cell as a drug widely known for the treatment of pancreatic cancer.

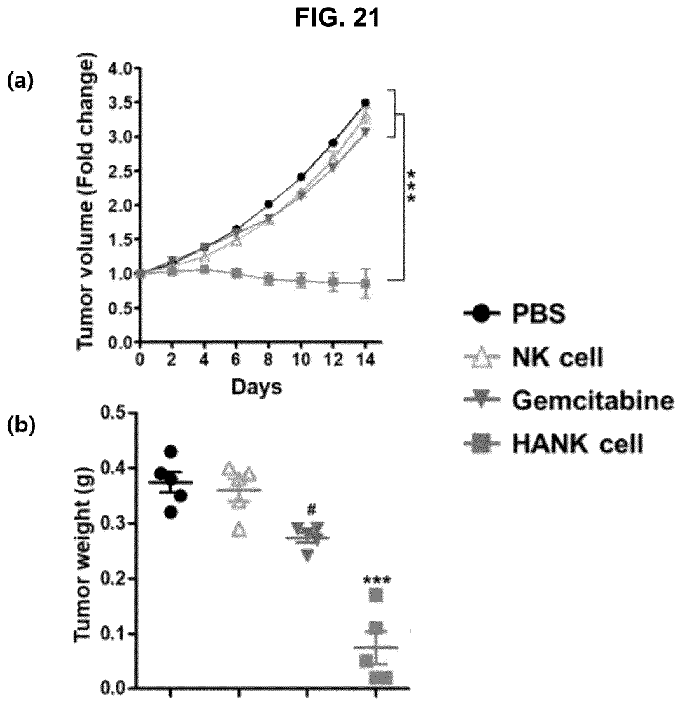

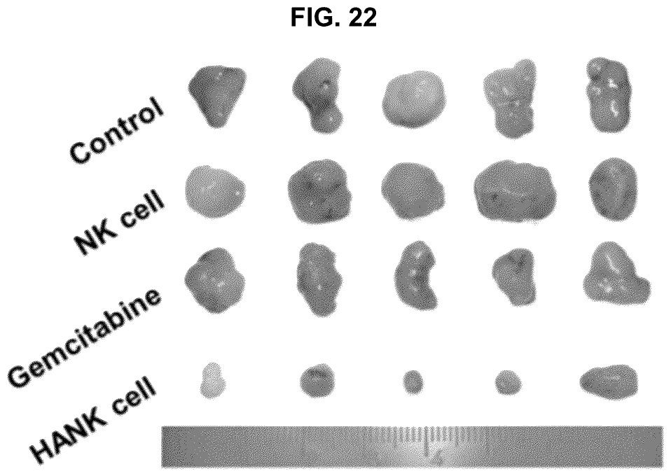

FIGS. 21 and 22 are views showing a change in volume, weight, and size of a tumor according to a progress of an experiment on pancreatic cancer.

Referring to FIGS. 21 and 22, a change in volume, weight, and size of the tumors in mice dosed with PBS, NK cell, gemcitabine, and HANK cell are shown. As can be seen in FIGS. 21 and 22, it can be confirmed that the volume, weight, and size of the tumors in mice dosed with HANK cell are significantly reduced.

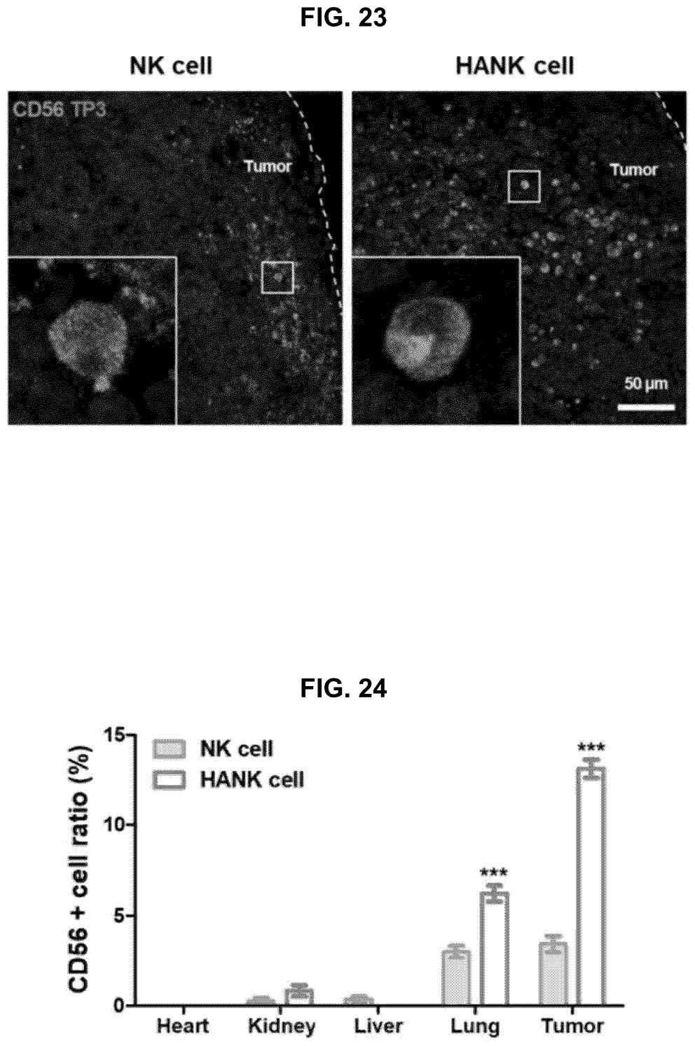

FIG. 23 is a view comparing a tumor penetration ability between NK cell and HANK cell.

Referring to FIG. 23, the results of using human-specific anti-CD56 to confirm the tumor penetration ability of NK cell and HANK cell are shown. As can be seen in FIG. 23, it can be confirmed that NK cell is mainly accumulated in a boundary of a tumor, whereas HANK cell is distributed in an entire region of the tumor. In other words, it can be seen that HANK cell has a significantly higher tumor penetration ability compared to NK cell.

FIG. 24 is a view for confirming a biodistribution of NK cell and HANK cell injected into experimental mice.

Referring to FIG. 24, the results of confirming a biodistribution after injecting NK cell and HANK cell into experimental mice are shown. Human-specific anti-CD56 was used to confirm the biodistribution.

As can be seen in FIG. 24, it could be confirmed that a very small amount of NK cell and HANK cell is detected in the heart, kidney, and liver, whereas a large amount of NK cell and HANK cell is detected in the lung and tumor. Accordingly, it can be seen that it is possible to effectively prevent pancreatic cancer from being transferred to the lung.

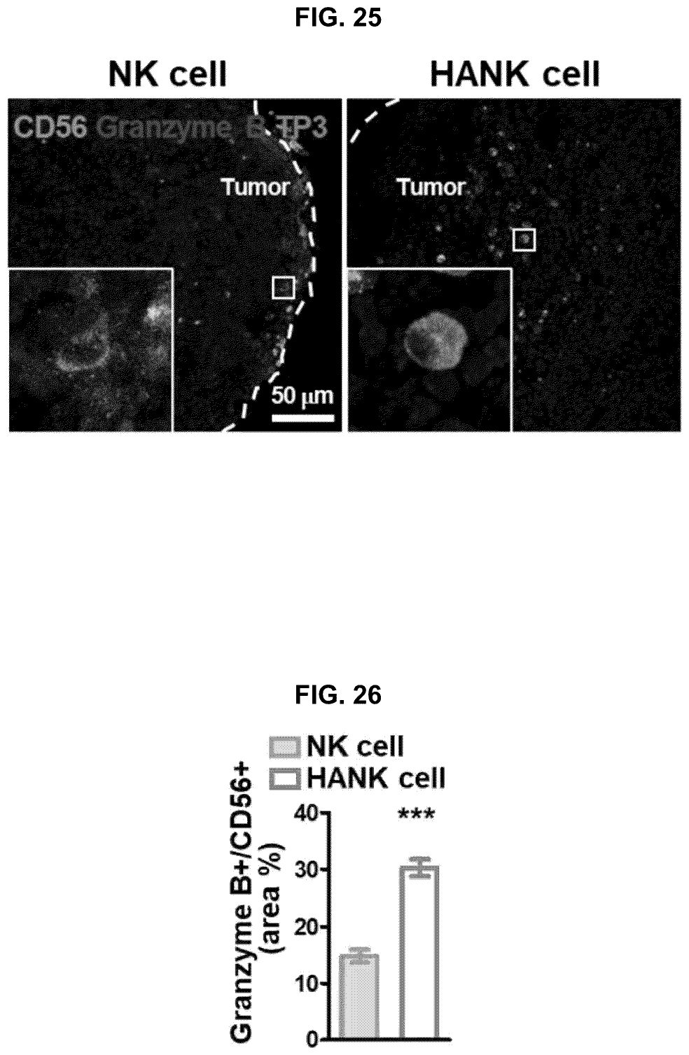

FIG. 25 and FIG. 26 are view comparing a cytotoxic granule spreading ability between NK cell and HANK cell.

Referring to FIGS. 25 and 26, the results of confirming an intratumoral distribution of Granzyme B, which is a cytotoxic granule secreted from NK cell and HANK cell, are shown. Human-specific anti-CD56 was used to confirm an intratumoral distribution of Granzyme B.

As can be seen in FIG. 25, it can be confirmed that Granzyme B secreted from NK cell is mainly distributed in a peripheral region of the tumor, whereas Granzyme B secreted from HANK cell is distributed in an entire region of the tumor. In addition, as can be seen in FIG. 26, it can be confirmed that a Granzyme B+/CD56+ cell area ratio (are %) is significantly higher in HANK cell compared to NK cell. Thus, it can be seen from FIGS. 25 and 26 that HA-PEG-Lipid promotes a cytotoxic granule secretion of NK cell.

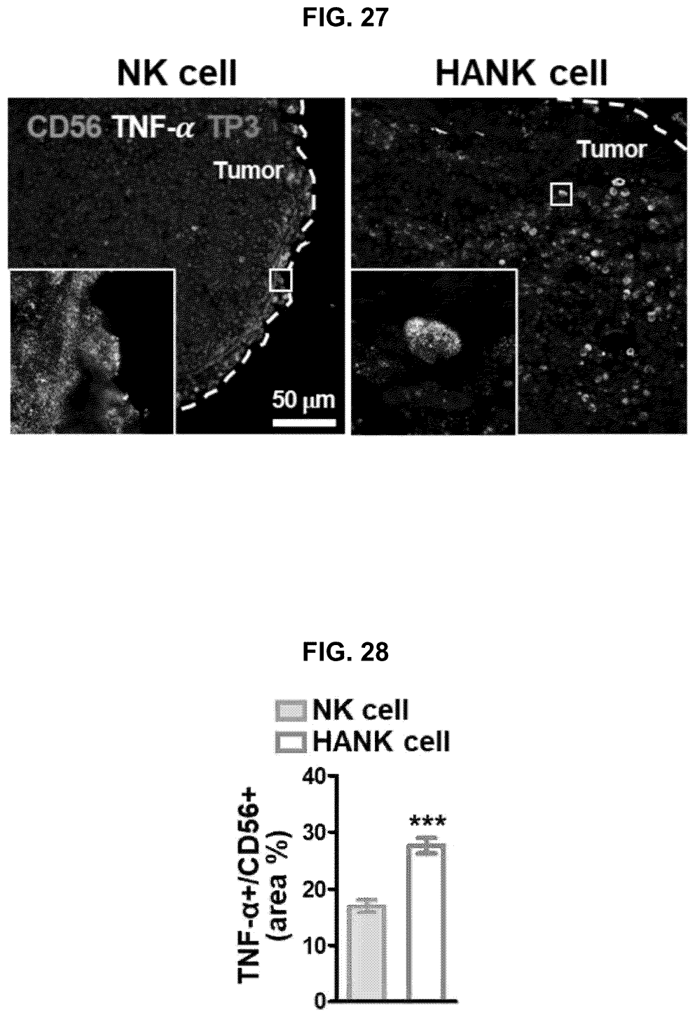

FIG. 27 and FIG. 28 are views comparing a cytokine spreading ability between NK cell and HANK cell.

Referring to FIGS. 27 and 28, the results of confirming an intratumoral distribution of TNF-α, which is a cytokine secreted from NK cell and HANK cell, are shown. Human-specific anti-CD56 was used to confirm an intratumoral distribution of TNF-α.

As can be seen in FIG. 27, it can be confirmed that TNF-α secreted from NK cell is mainly distributed in a peripheral region of a tumor, whereas Granzyme B secreted from HANK cell is distributed in an entire region of the tumor. In addition, as can be seen in FIG. 28, it could be confirmed that a TNF-α/CD56+ cell area ratio (are %) is significantly higher in HANK cell compared to NK cell. Thus, it can be seen from FIGS. 27 and 28 that HA-PEG-Lipid promotes a cytokine secretion of NK cell.

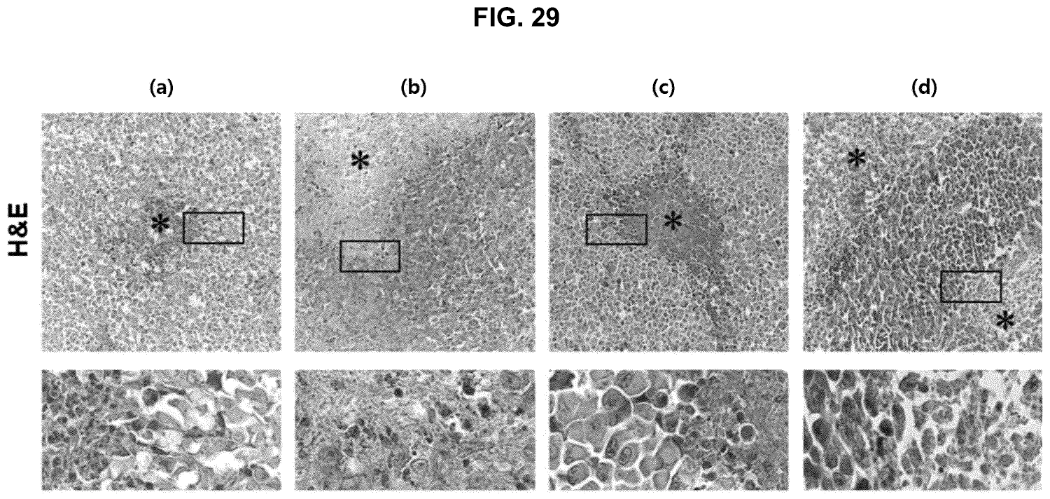

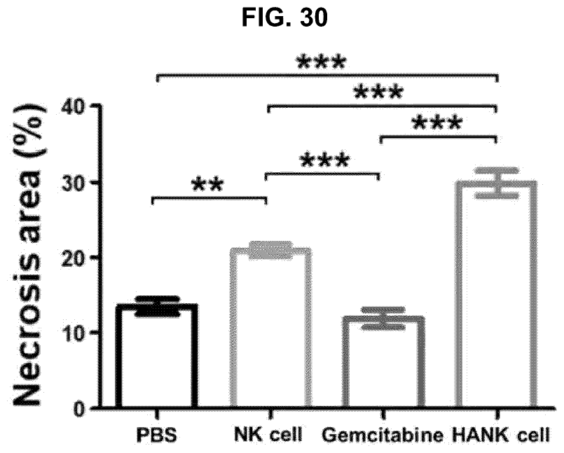

FIG. 29 and FIG. 30 are views comparing a tumor necrosis ability among PBS, NK cell, gemcitabine, and HANK cell.

Referring to FIG. 29, a degree of tumor necrosis is shown in such a way that hematoxylin and eosin (H&E) are stained on subcutaneous tumors of experimental mice injected with PBS (a), NK cell (b), gemcitabine (c), and HANK cell (d), and referring to FIG. 30, a subcutaneous tumor necrosis region (necrosis area, %) of experimental mice injected with PBS, NK cell, gemcitabine, and HANK cell is quantified and shown.

As can be seen in FIGS. 29 and 30, it can be confirmed that HANK cell exhibits a significantly improved tumor necrosis ability compared to NK cell and gemcitabine.

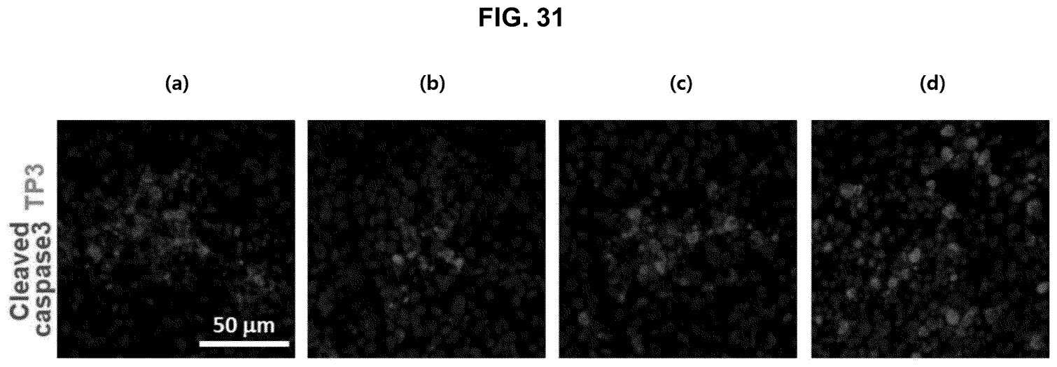

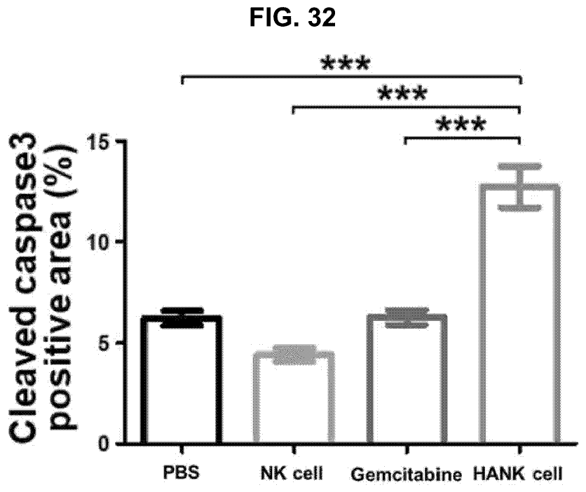

FIG. 31 and FIG. 32 are views comparing an apoptotic region of tumor masses via PBS, NK cell, gemcitabine, and HANK cell.

Referring to FIG. 31, a result of indicating an apoptotic region of a tumor mass as a cleaved caspase3 in experimental mice injected with PBS (a), NK cell (b), gemcitabine (c), and HANK cell (d) is shown, and referring to FIG. 32, a positive area of the cleaved caspase3 in experimental mice injected with PBS (a), NK cell (b), gemcitabine (c), and HANK cell (d) is quantified and shown.

As can be seen in FIG. 31, it can be confirmed that HANK cell shows a stronger expression of cleaved caspase3 compared to NK cell and gemcitabine. In addition, as can be seen in FIG. 32, it can be confirmed that HANK cell has a significantly higher positive area ratio of cleaved caspase3 compared to NK cell and gemcitabine.

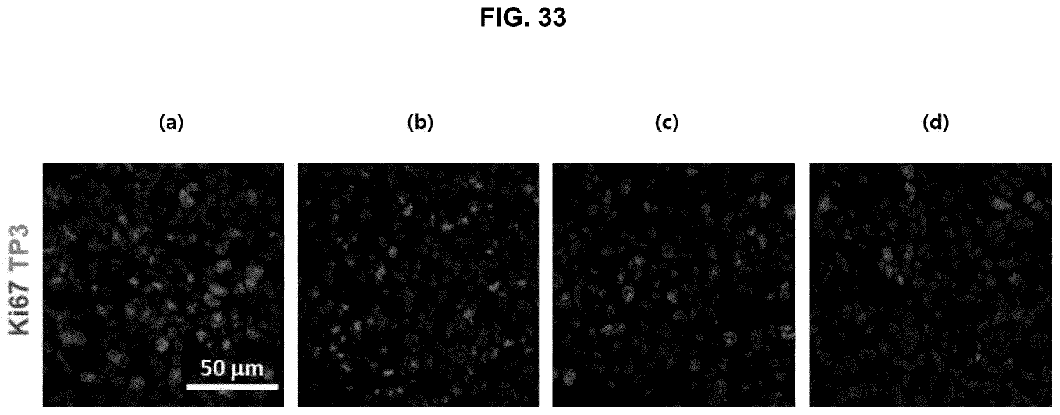

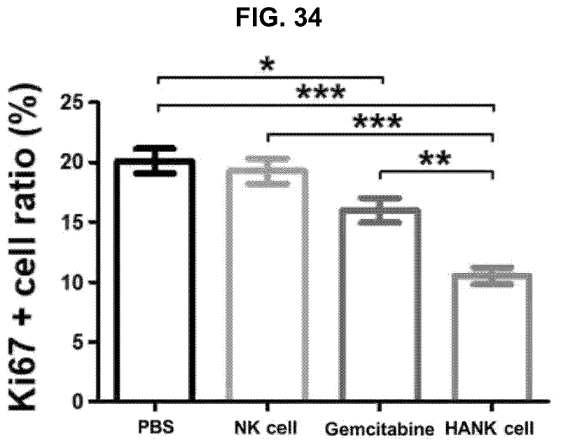

FIG. 33 and FIG. 34 are view comparing a cell proliferation inhibitory ability among PBS, NK cell, gemcitabine, and HANK cell.

Referring to FIG. 33, a result of confirming a degree of cell proliferation by staining Ki67 on subcutaneous tumors of experimental mice injected with PBS (a), NK cell (b), gemcitabine (c), and HANK cell (d) is shown, and referring to FIG. 34, a proportion of Ki67 positive cells in experimental mice injected with PBS (a), NK cell (b), gemcitabine (c), and HANK cell (d) is quantified and shown.

As can be seen in FIG. 33, it can be confirmed that HANK cell exhibits a significantly lower Ki67 expression compared to NK cell and gemcitabine. In addition, as can be seen in FIG. 34, it can be confirmed that HANK cell has a significantly lower cell proliferation region compared to NK cell and gemcitabine. In other words, it can be seen that HANK cell has a significantly higher cell proliferation inhibitory ability compared to NK cell and gemcitabine.

### Preparation of Polymer Compound (HA-PEG-DSPE) According to Experimental Example 2

100 mg of hyaluronic acid (Mw 60 k, 10 mg/mL, 1 equivalent), 0.075 mmol of 1-ethyl-3-(3-dimethylaminopropyl) carbodiimide (EDC), and 0.075 mmol of N-hydroxy succinimide (NHS) were dissolved in 8 mL of DI water and stirred at room temperature for six hours to prepare a base solution.

After that, a solution in which 0.05 mmol of DSPE-PEG-NH₂ was dissolved in 2 mL of dimethylformamide (DMF), as well as 4-dimethylaminopyridine (DMAP) were added to the base solution described above, and then stirred at room temperature for 48 hours.

Finally, the stirred reaction product was dialyzed at DI water for three days (MWCO 12-14 kDa) to prepare a HA-PEG-DSPE polymer compound.

### Preparation of Polymer Compound (HA-PEG-DMPE) According to Experimental Example 2

100 mg of hyaluronic acid (Mw 60 k, 10 mg/mL, 1 equivalent), 0.075 mmol of 1-ethyl-3-(3-dimethylaminopropyl) carbodiimide (EDC), and 0.075 mmol of N-hydroxy succinimide (NHS) were dissolved in 8 mL of DI water and stirred at room temperature for six hours to prepare a base solution.

After that, a solution in which 0.05 mmol of DMPE-PEG-NH₂ was dissolved in 2 mL of dimethylformamide (DMF) and 4-dimethylaminopyridine (DMAP) were added to the base solution described above, and then stirred at room temperature for 48 hours.

Finally, the stirred reaction product was dialyzed at DI water for three days (MWCO 12-14 kDa) to prepare a HA-PEG-DMPE polymer compound.

### Preparation of Polymer Compound (HA-PEG-Chol) According to Experimental Example 2

100 mg of hyaluronic acid (Mw 60 k, 10 mg/mL, 1 equivalent), 0.075 mmol of 1-ethyl-3-(3-dimethylaminopropyl) carbodiimide (EDC), and 0.075 mmol of N-hydroxy succinimide (NHS) were dissolved in 8 mL of DI water and stirred at room temperature for six hours to prepare a base solution.

After that, a solution in which 0.05 mmol of cholesterol (Chol)-PEG-NH₂ was dissolved in 2 mL of dimethylformamide (DMF), as well as 4-dimethylaminopyridine (DMAP) were added to the base solution described above, and then stirred at room temperature for 48 hours.

Finally, the stirred reaction product was dialyzed at DI water for three days (MWCO 12-14 kDa) to prepare a HA-PEG-Chol polymer compound.

### Experimental Example 2-1: Characterization of Polymer Compound

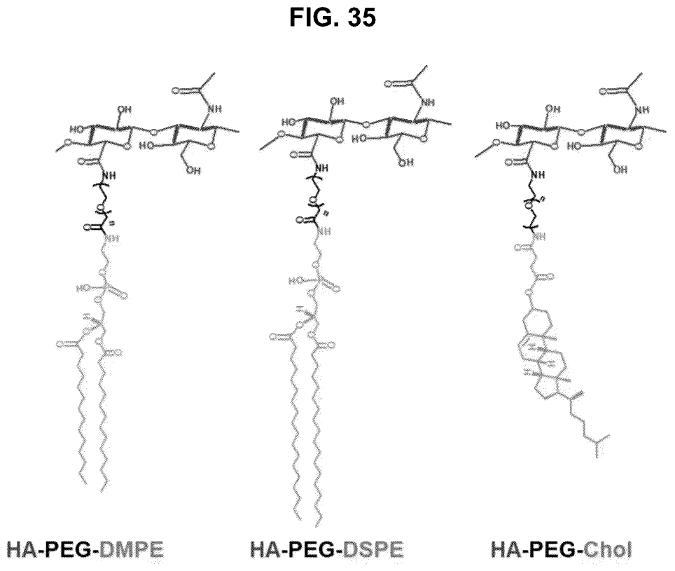

FIG. 35 is a view for describing a structural formula of a polymer compound according to Experimental Example 2 of the present invention.

Referring to FIG. 35, the structural formulas for the polymer compounds HA-PEG-DSPE, HA-PEG-DMPE, and HA-PEG-Chol according to the experimental example may be confirmed.

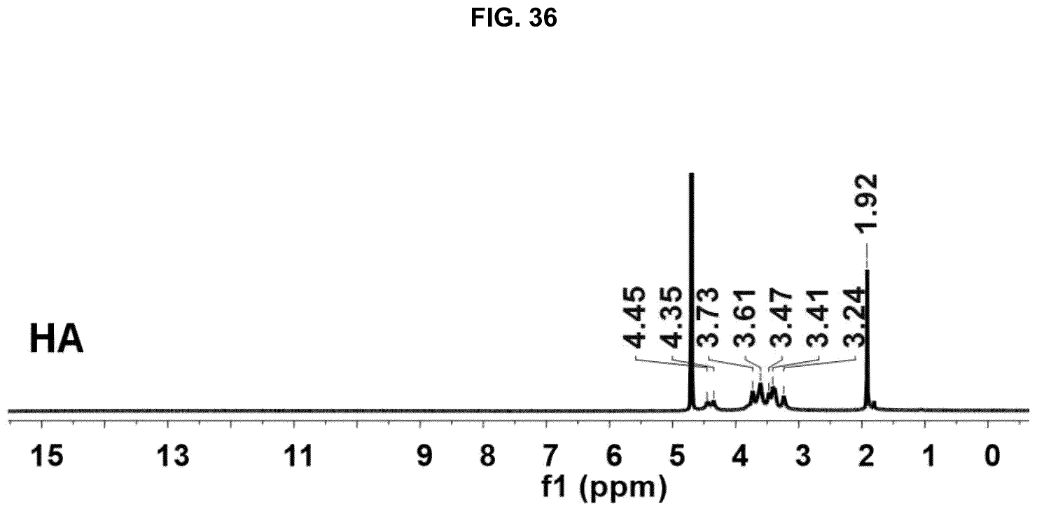

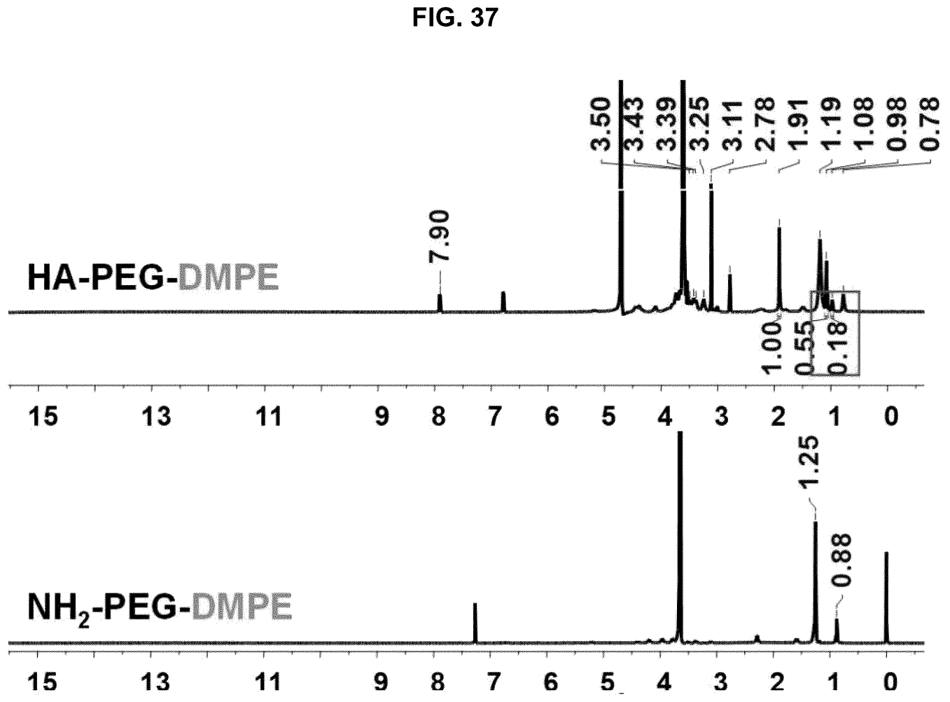

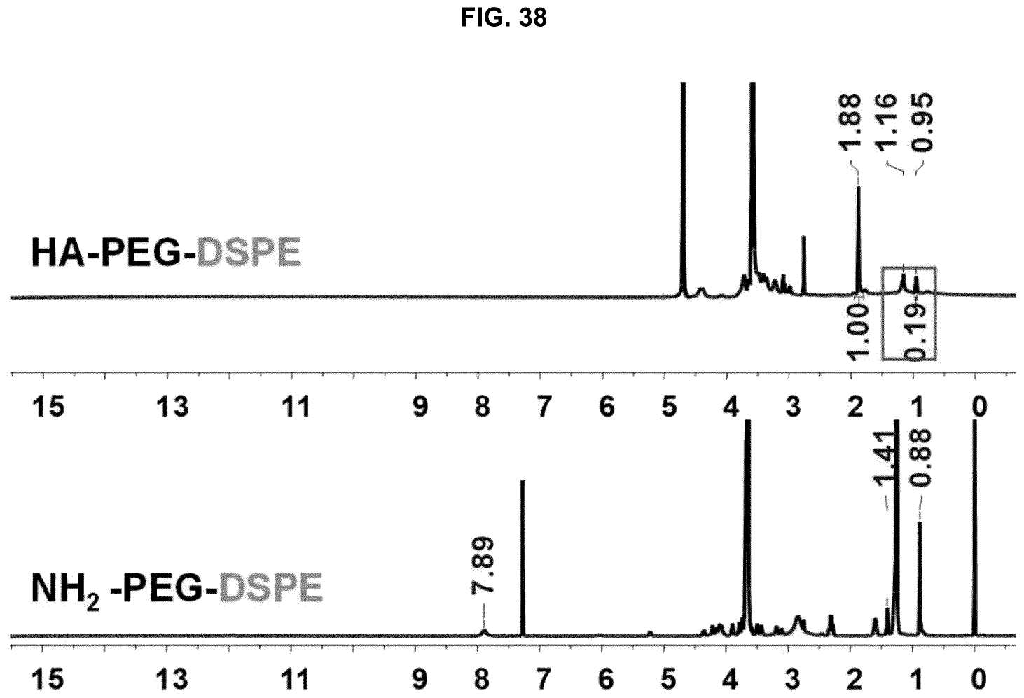

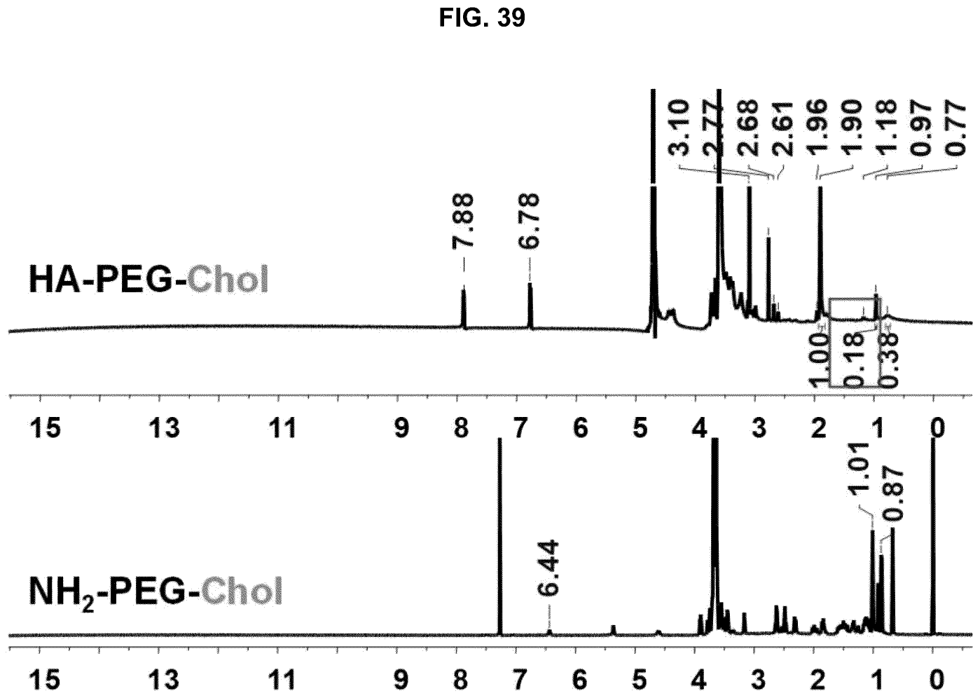

FIG. 36 is a view for describing a result of proton NMR analysis on hyaluronic acid (HA), FIG. 37 is a view for describing a result of proton NMR analysis on a HA-PEG-DMPE polymer compound, FIG. 38 is a view for describing a result of proton NMR analysis on a HA-PEG-DSPE polymer compound, and FIG. 39 is a view for describing a result of proton NMR analysis on an HA-PEG-Chol polymer compound.

Referring to FIG. 36, in the case of hyaluronic acid (HA), it can be confirmed that peaks are observed at 1.92 ppm, 3.24 ppm, 3.41 ppm, 3.47 ppm, 3.61 ppm, 3.73 ppm, 4.35 ppm, and 4.45 ppm as a result of proton NMR analysis.

Referring to FIG. 37, a result of proton NMR analysis on HA-PEG-DMPE and NH₂-PEG-DMPE are shown. As can be seen in FIG. 37, it can be confirmed that HA-related peaks are observed as HA is conjugated to NH₂-PEG-DMPE. Specifically, in the case of HA-PEG-DMPE, it can be confirmed that peaks are observed at 0.18 ppm, 0.55 ppm, 0.78 ppm, 0.98 ppm, 1.00 ppm, 1.08 ppm, 1.19 ppm, 1.91 ppm, 2.78 ppm, 3.11 ppm, 3.25 ppm, 3.39 ppm, 3.43 ppm, 3.50 ppm, and 7.90 ppm as a result of proton NMR analysis.

Referring to FIG. 38, a result of proton NMR analysis on HA-PEG-DSPE and NH₂-PEG-DSPE are shown. As can be seen in FIG. 38, it can be confirmed that HA-related peaks are observed as HA is conjugated to NH₂-PEG-DSPE. Specifically, in the case of HA-PEG-DSPE, it can be confirmed that peaks are observed at 0.19 ppm, 0.95 ppm, 1.00 ppm, 1.16 ppm, and 1.88 ppm as a result of proton NMR analysis.

Referring to FIG. 39, a result of proton NMR analysis on HA-PEG-Chol and NH₂-PEG-Chol are shown. As can be seen in FIG. 39, it can be confirmed that HA-related peaks are observed as HA is conjugated to NH₂-PEG-Chol. Specifically, in the case of HA-PEG-Chol, it can be confirmed that peaks are observed at 0.18 ppm, 0.38 ppm, 0.77 ppm, 0.97 ppm, 1.00 ppm, 1.18 ppm, 1.90 ppm, 1.96 ppm, 2.61 ppm, 2.68 ppm, 2.77 ppm, 3.10 ppm, 6.78 ppm, and 7.88 ppm as a result of proton NMR analysis.

In addition, as can be seen in FIGS. 37 to 39, in the case of NH₂-PEG-DMPE and NH₂-PEG-Chol, it can be confirmed that a peak is observed in the range of 6 to 9 ppm, but in the case of NH₂-PEG-DSPE, it can be confirmed that a peak is not observed in the range of 6 to 9 ppm.

### Experimental Example 2-2: Analysis of NK Cell Bound to Polymer Compound Properties

The polymer compound (HA-PEG-Lipid) according to the experimental example was dissolved in MEM alpha, and 5×10⁵ NK-92 mi NK cells were evenly mixed with 100 μL of a solution, and then reacted at room temperature for 30 minutes to prepare cells in which the polymer compound (HA-PEG-Lipid) according to the experimental example was bound to NK cells.

A cell in which HA-PEG-DMPE is bound to NK cell may be defined as HADM-NK cell, a cell in which HA-PEG-DSPE is bound to NK cell may be defined as HADS-NK cell, and a cell in which HA-PEG-Chol is bound to NK cell may be defined as HACH-NK cell.

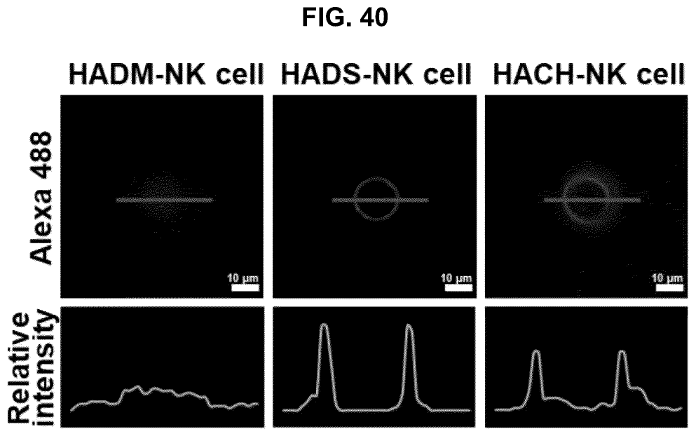

FIG. 40 is a view for confirming an endocytosis phenomenon of HADM-NK cell, HADS-NK cell, and HACH-NK cell.

Referring to FIG. 40, a result of confirming the occurrence of an endocytosis phenomenon is shown in such a way that HADM-NK cell, HADS-NK cell, and HACH-NK cell are treated with Alex Flour 488 hydrazide (fluorescent dye), respectively. As can be seen in FIG. 40, it can be confirmed that the endocytosis phenomenon does not occur in the case of HADS-NK cell and HACH-NK cell, but the endocytosis phenomenon occurs in the case of HADM-NK cell.

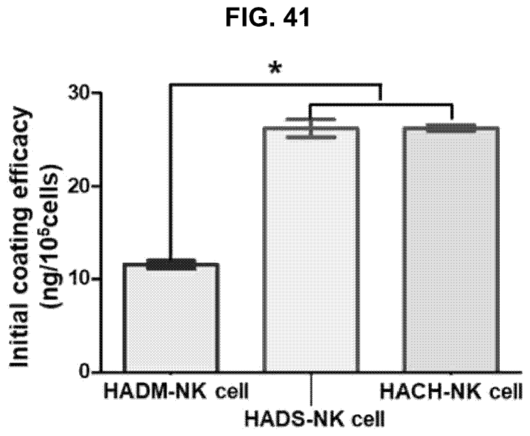

FIG. 41 is a view for describing a coating efficacy of HADM-NK cell, HADS-NK cell, and HACH-NK cell.

Referring to FIG. 41, the coating efficacy (the ratio of the polymer compound bound to NK cell) of HADM-NK cell, HADS-NK cell, and HACH-NK cell is quantitatively shown. As can be seen in FIG. 41, it can be confirmed that the coating efficacy of HADM-NK cell is significantly lower compared to HADS-NK cell and HACH-NK cell.

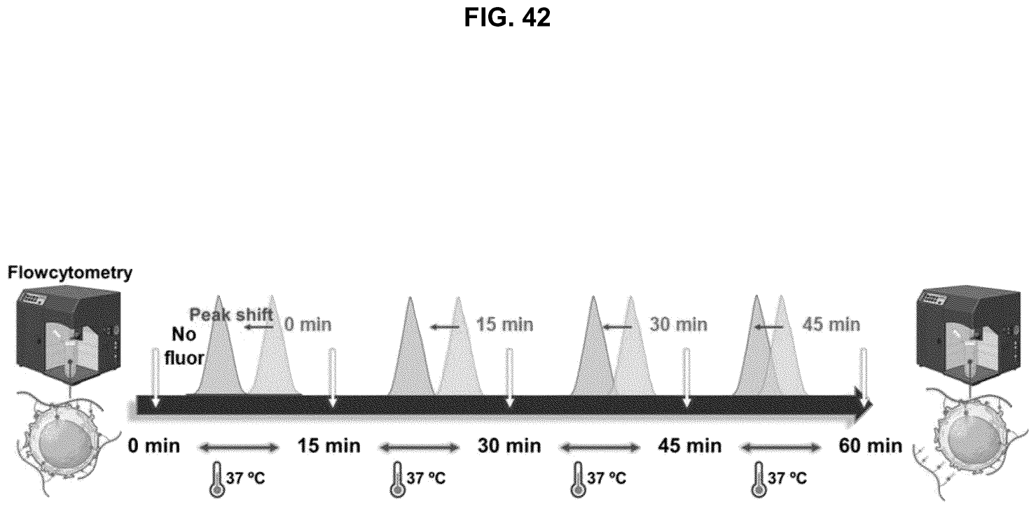

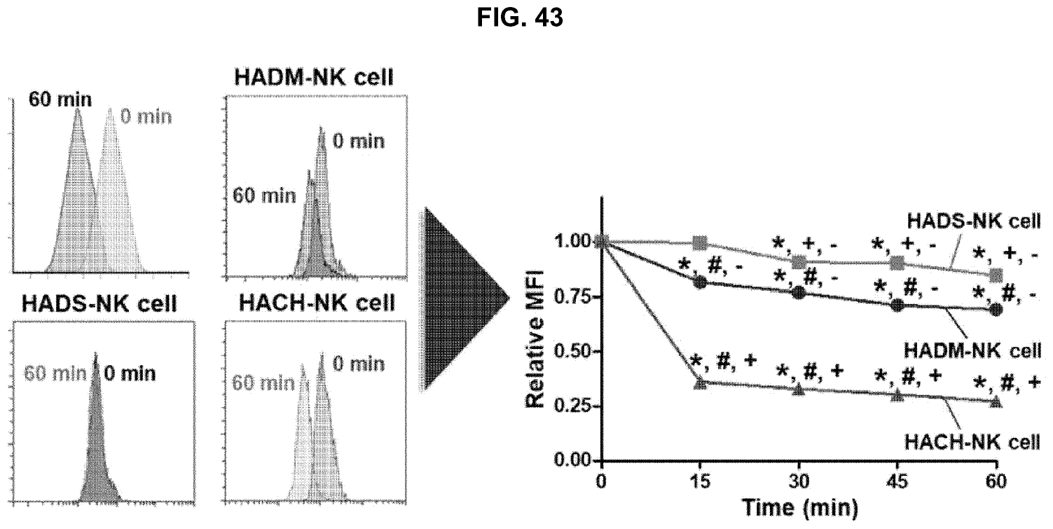

FIG. 42 is a schematic view showing a flow cytometry for measuring a fluorescence intensity of HADM-NK cell, HADS-NK cell, and HACH-NK cell, and FIG. 43 is a view quantitatively showing a fluorescence intensity measured by the method of FIG. 42.

Referring to FIGS. 42 and 43, the fluorescence intensity of HADM-NK cell, HADS-NK cell, and HACH-NK cell were measured by flow cytometry to confirm how long a polymer compound bound to the surface of NK cell is bound to NK cell (binding persistence). As can be seen in FIG. 43, in the case of HADM-NK cell and HADS-NK cell, it can be confirmed that the polymer compound bound to NK-cell lasts for a relatively long time, whereas in the case of HACH-NK cell, it can be confirmed that 63.8% is removed within 60 minutes after the polymer compound (HA-PEG-Chol) is rapidly removed within the initial 15 minutes after binding.

As a result, as can be seen in Experimental Example 2-2 (FIGS. 40 to 43), in the case of HA-PEG-DMPE, it can be seen that the binding efficiency with NK cell is significantly low, and in the case of HA-PEG-Chol, it can be confirmed that the binding efficiency with NK cell is high, but the binding persistence is significantly low, and thus making it unsuitable for use as a hydrophobic moiety for binding to NK cell. On the contrary, it can be seen that HA-PEG-DSPE has high binding efficiency with NK cell as well as high binding persistence, and thus may be easily used as a hydrophobic moiety for binding with NK cell.

### Experimental Example 2-3: Evaluation of Effect of Polymer Compound on NK Cell

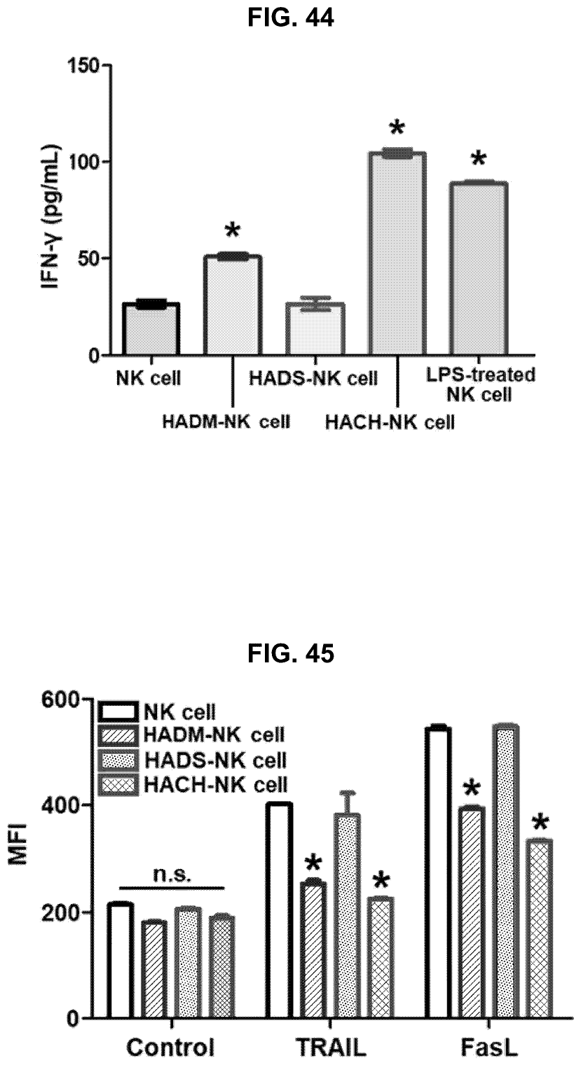

FIG. 44 is a view comparing an amount of cytokine secretion among NK cell, HADM-NK cell, HADS-NK cell, HACH-NK cell, and LPS-treated NK cell.

Referring to FIG. 44, the amount of cytokine (IFN-Y) secretion of NK cell, HADM-NK cell, HADS-NK cell, HACH-NK cell, and LPS-treated NK cell is measured and shown. NK cell may refer to a natural killer cell which is not bound to a polymer compound, and an LPS-treated NK cell may mean NK cell treated with LPS which induces the secretion of IFN-Y.

As can be seen in FIG. 44, it can be confirmed that HADM-NK cell and HACH-NK cell excessively secrete IFN-Y compared to NK cell. When IFN-Y is excessively secreted, inflammation may be induced and tumor metastasis may occur, and thus, it can be seen that HADM-NK cell and HACH-NK cell are unsuitable for cancer treatment. On the contrary, it can be seen that HADS-NK cell has a similar amount of IFN-Y secretion compared to NK cell. In other words, it can be confirmed that HA-PEG-DSPE does not affect the secretion of appropriate cytokines (IFN-Y), which is one of the unique roles of NK cell.

FIG. 45 is a view for comparing a content of TRAIL ligands and FasL ligands present on surfaces among NK cell, HADM-NK cell, HADS-NK cell, and HACH-NK cell.

Referring to FIG. 45, the result of making an MFI analysis by flow cytometry after treating HADM-NK cell, HADS-NK cell, and HACH-NK cell with TRAIL antibody and FasL antibody, respectively, and then detecting TRAIL and FasL present on the surface of each cell is shown. FasL and TRAIL may be major surface ligands of NK cell which may recognize target cancer cells and induce direct killing, and as can be seen in FIG. 45, it can be confirmed that HADS-NK cell shows similar MFI values of TRAIL and FasL compared to NK cell, whereas HADM-NK cell and HACH-NK cell show reduced MFI values of TRAIL and FasL compared to NK cell.

In other words, it can be seen that HA-PEG-DSPE does not substantially affect TRAIL ligand and FasL ligand availability of NK cell even when bound to NK cell, whereas HA-PEG-DMPE and HA-PEG-Chol interfere with TRAIL ligand and FasL ligand availability of NK cell.

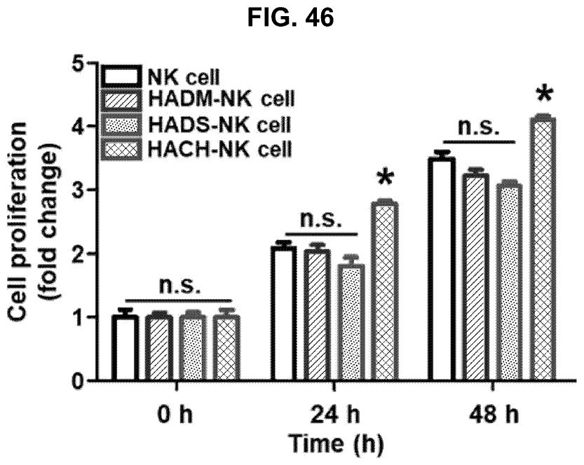

FIG. 46 is a view for comparing the viability and cell proliferation ability among NK cell, HADM-NK cell, HADS-NK cell, and HACH-NK cell.

Referring to FIG. 46, the viability and cell proliferation ability of NK cell, HADM-NK cell, HADS-NK cell, and HACH-NK cell are compared and shown. As can be seen in FIG. 46, it can be confirmed that all of HADM-NK cell, HADS-NK cell, and HACH-NK cell show similar viability and cell proliferation ability compared to NK cell. In other words, it can be seen that all of HA-PEG-DMPE, HA-PEG-DSPE, and HA-PEG-Chol do not affect the viability and cell proliferation ability of NK cell.

### Experimental Example 2-4: Anticancer Mechanism of NK Cell Bound to Polymer Compound

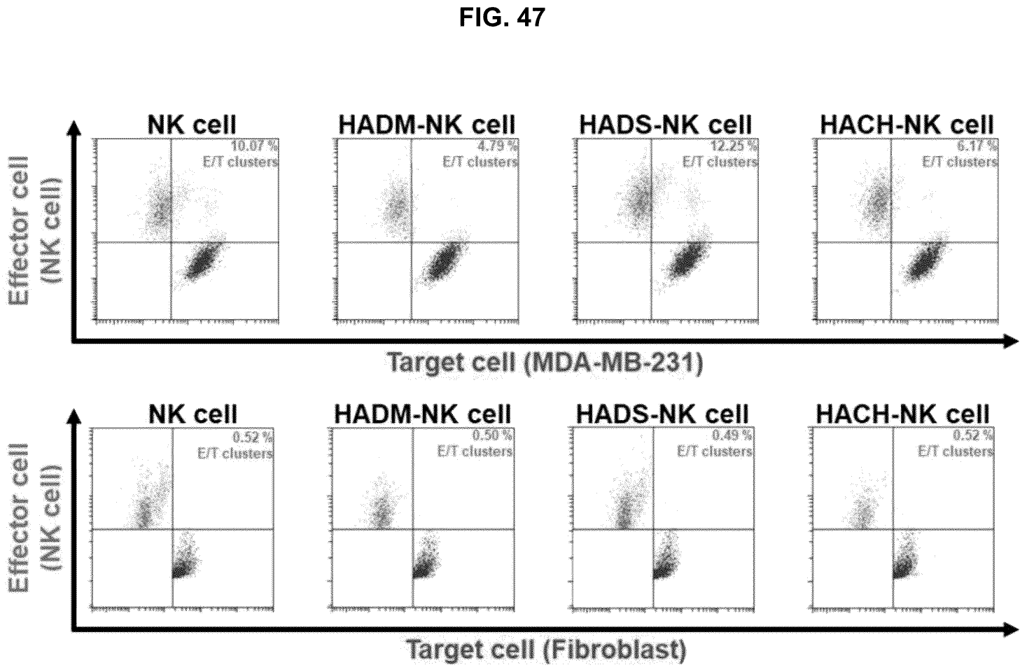

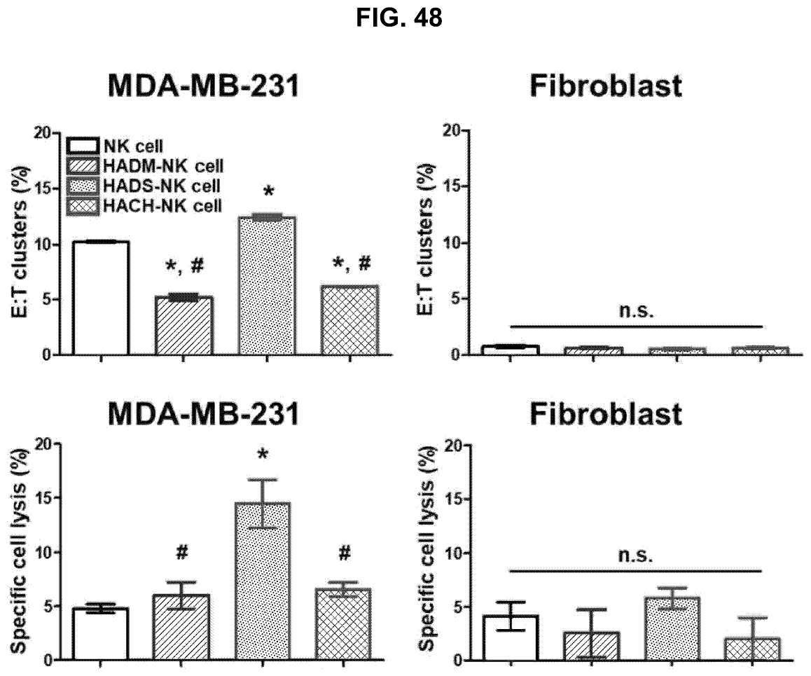

FIGS. 47 and 48 are views for describing a target recognition ability and anticancer efficacy of NK cell, HADM-NK cell, HADS-NK cell, and HACH-NK cell.

Referring to FIGS. 47 and 48, each target cell recognition ability of NK cell, HADM-NK cell, HADS-NK cell, and HACH-NK cell is measured and shown. Specifically, MDA-MB-231, which was a CD44 positive cancer cell line, and fibroblast, which was a CD44 negative cancer cell line, were used as a target cell. In addition, the target recognition ability was assessed by quantification of effector/target (E/T) cluster. In addition to the target recognition ability, the cell lysis rate (cell lysis, %) of target cell was measured, which is shown in FIG. 48.

As can be seen in FIGS. 47 and 48, in the case of HADM-NK cell and HACH-NK cell, it can be confirmed that the target recognition ability (E:T clusters, %) for MDA-MB-231 is relatively low compared to NK cell and HADS-NK cell. In addition, it could be confirmed that HADS-NK cell has a significantly higher cell lysis rate (cell lysis, %) compared to NK cell, HADM-NK cell, and HACH-NK cell. Furthermore, it could be confirmed that NK cell, HADM-NK cell, HADS-NK cell, and HACH-NK cell do not respond to fibroblast, which is a CD44 negative cancer cell line. As a result, it can be seen that HA-PEG-DSPE may bind to NK cell to selectively recognize and then effectively kill CD44-positive cancer cell lines.

### Preparation of Polymer Compound (HA-PEG-DSPE) According to Experimental Example 3

100 mg of hyaluronic acid (Mw 60 k, 10 mg/mL, 1 equivalent), 0.075 mmol of 1-ethyl-3-(3-dimethylaminopropyl) carbodiimide (EDC), and 0.075 mmol of N-hydroxy succinimide (NHS) were dissolved in 8 mL of DI water and stirred at room temperature for six hours to prepare a base solution.

After that, a solution in which 0.05 mmol of DSPE-PEG-NH₂ was dissolved in 2 mL of dimethylformamide (DMF), as well as 4-dimethylaminopyridine (DMAP) were added to the base solution described above, and then stirred at room temperature for 48 hours.

Finally, the stirred reaction product was dialyzed at DI water for three days (MWCO 12-14 kDa) to prepare a HA-PEG-DSPE polymer compound. In addition, the preparation method described above is shown in FIG. 2.

Furthermore, a polymer compound was prepared using PEGs (0 k, 0.6 k, 2 k, 5 k) having different lengths, and the prepared polymer compounds may be represented by PEG 0, PEG 600, PEG 2000, and PEG 5000 in describing the following experimental examples.

Moreover, when 2 k-long PEG was used, a degree of substitution (DS) defined as a value obtained by calculating an amount of the DSPE linked to the hyaluronic acid through an NMR analysis varied for preparation at 7%, 18%, and 26%, and the prepared polymer compounds were represented by PEG 2000 7%, PEG 2000 18%, and PEG 2000 26% in describing the following experimental examples. Specifically, HA-PEG-DSPEs having different DSs were prepared by varying a content of DSPE-PEG-NH₂ used in the above-described preparation process. The HA-PEG-DSPE having a DS of 7% was prepared using 70 μg of DSPE-PEG-NH₂, the HA-PEG-DSPE having a DS of 18% was prepared using 140 μg of DSPE-PEG-NH₂, and the HA-PEG-DSPE having a DS of 26% was prepared using 280 μg of DSPE-PEG-NH₂.

### Experimental Example 3-1: Characterization of Polymer Compound

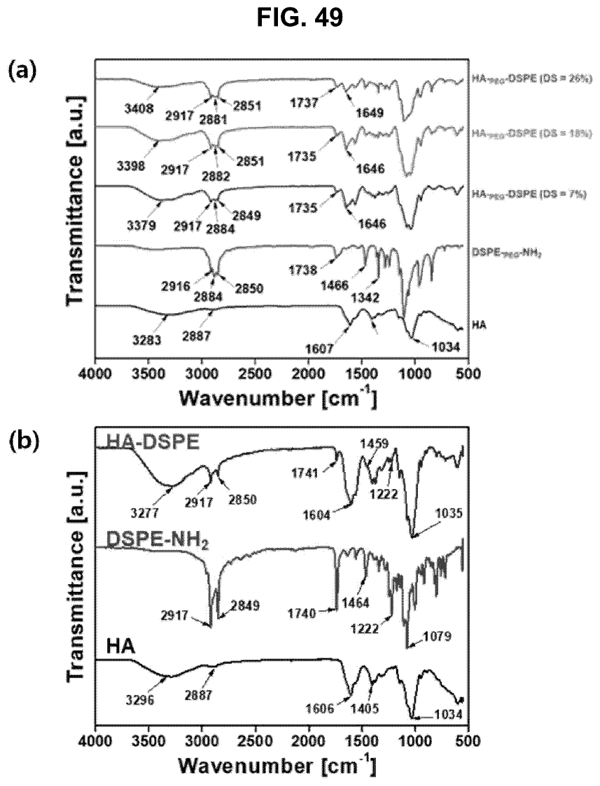

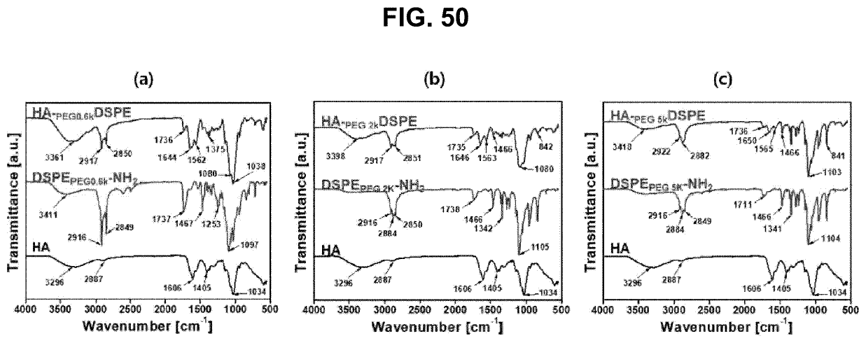

FIGS. 49 and 50 are views showing a result of FT-IR analysis on polymer compounds according to experimental examples of the present invention.

Referring to (a) of FIG. 49, a result of FT-IR analysis on each of hyaluronic acid (HA), DSPE-PEG-NH₂, HA-PEG (2000, 7%)-DSPE (DS=7%), HA-PEG (2000, 18%)-DSPE (DS=18%), and HA-PEG (2000, 26%)-DSPE (DS=26%) is shown. Referring to (b) of FIG. 49, a result of FT-IR analysis on each of hyaluronic acid (HA), DSPE-PEG-NH₂, and HA-PEG-DSPE is shown. Referring to (a) of FIG. 50, a result of FT-IR analysis on each of hyaluronic acid (HA), DSPE-PEG_{0.6k}-NH₂, and HA-PEG_{0.6k}-DSPE is shown. Referring to (b) of FIG. 50, a result of FT-IR analysis on each of hyaluronic acid (HA), DSPE-PEG₂ₖ-NH₂, and HA-PEG₂ₖ-DSPE is shown. And referring to (c) of FIG. 50, a result of FT-IR analysis on each of hyaluronic acid (HA), DSPE-PEG₅ₖ-NH₂, and HA-PEG₅ₖ-DSPE is shown. As can be seen in FIGS. 49 and 50, it can be confirmed that DSPE and HA are easily bound to PEG (0.6 k, 2 k, 5 k) having different lengths.

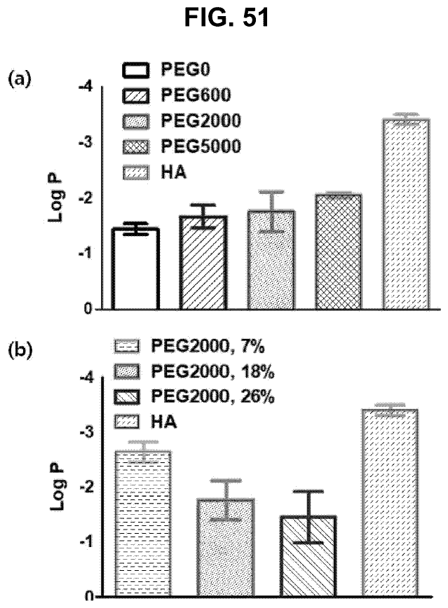

FIG. 51 is a view showing a result of hydrophobic analysis on polymer compounds according to experimental examples of the present invention.

Referring to (a) of FIG. 51, a result of measuring a Log P value to confirm hydrophobicity for each of HA-PEG0-DSPE, HA-PEG600-DSPE, HA-PEG2000-DSPE, HA-PEG5000-DSPE, and hyaluronic acid (HA) is shown, and referring to (b) of FIG. 51, a result of measuring a Log P value to confirm hydrophobicity for each of HA-PEG2000, 7%-DSPE, HA-PEG2000, 18%-DSPE, HA-PEG2000, 26%, and hyaluronic acid (HA) is shown. The Log P value was calculated through <Equation 1> below.

Log P=(solute)Octanol/(solute)water <Equation 1>

As can be seen in (a) of FIG. 51, it could be confirmed that as a length of PEG increases (0->600->2000->5000), a Log P value increases, and thus the hydrophobicity decreases. In addition, as can be seen in (b) of FIG. 51, it could be confirmed that the Log P value decreases as the DS value increases (7%->18%->26%). In other words, it can be seen that the hydrophobicity decreases as the content of lipid decreases (as the DS value decreases).

### Experimental Example 3-2: Analysis of NK Cell Surface Modification Efficiency of Polymer Compound

A polymer compound (HA-PEG-DSPE) was dissolved in MEM alpha at a concentration of 1.0 mg/mL, and 5×10⁵ NK92-mi NK cells were evenly mixed with 100 μL of solution. The surface of NK cell was modified (HA-PEG-DSPE bound to NK cell) at room temperature for 30 minutes and then washed twice with MEM alpha. Cells were then lysed with 250 μL of RIPA buffer and stored at 4° C. for 30 minutes. Finally, 250 μL of distilled water was added, diluted, and transferred to 96-well to measure fluorescence intensity at 480/535 nm.

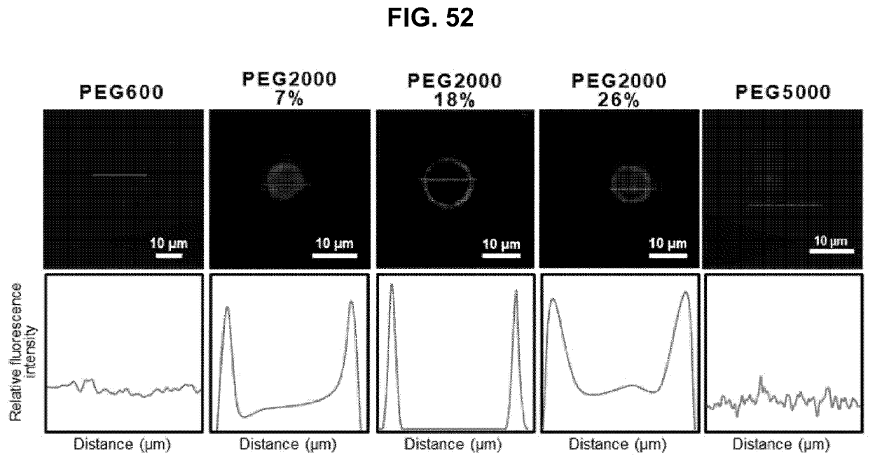

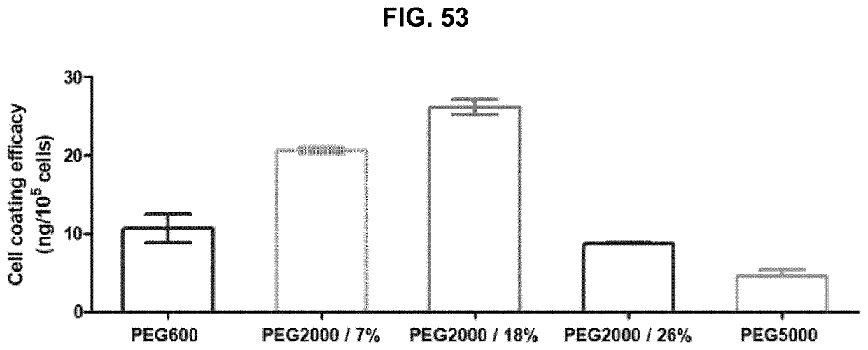

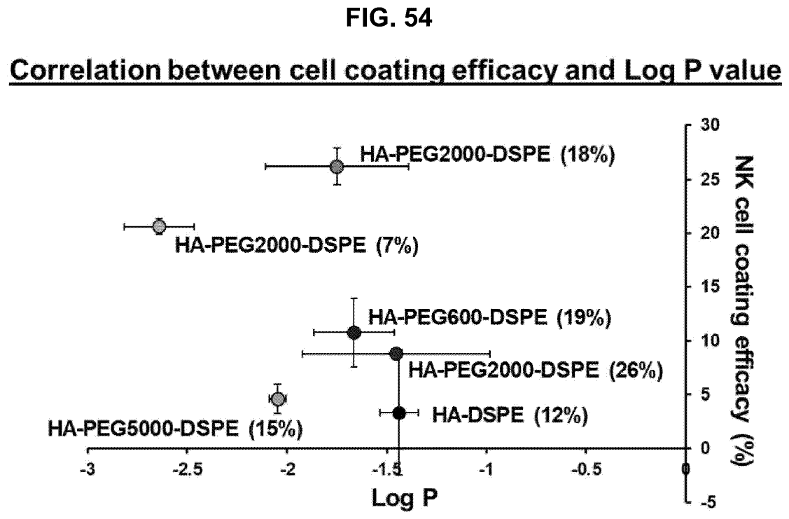

FIG. 52 is an image of measured fluorescence intensity and a graph of measured fluorescence intensity with regard to cells in which the polymer compounds are bound to NK cells according to experimental examples of the present invention, FIG. 53 is a graph showing a quantitative comparison of the NK cell surface modification efficiency among polymer compounds according to experimental examples of the present invention, and FIG. 54 is a graph summarizing an NK cell surface modification efficiency of the polymer compounds with respect to a log P value according to experimental examples of the present invention.

As can be seen in FIG. 52, in the case of the polymer compound using PEG600 and the polymer compound using PEG5000, it can be confirmed that fluorescence intensity is not substantially measured. On the contrary, in the case of the polymer compound using PEG2000, it can be confirmed that strong fluorescence intensity is measured on the surface of NK cell. In other words, it can be confirmed that the polymer compound using PEG2000 is well immobilized to the surface of NK cell, whereas the polymer compound using PEG600 and PEG5000 is not immobilized to the surface of NK cell. Accordingly, it can be seen that the length of PEG needs to be controlled to be greater than 0.6 k and less than 5 k in order to improve the cancer cell killing efficiency of NK cell through the HA-PEG-DSPE polymer compound bound to NK cell.

As can be seen in FIG. 53, it can be confirmed that the polymer compound using PEG2000 has higher coating efficacy (cell coating efficacy, ng/10⁵ cells) of NK cell compared to the polymer compound using PEG600 and PEG5000. In particular, it can be confirmed that the coating efficacy of NK cell increases when the DS increases from 7% to 18% in the polymer compound using PEG2000, but the coating efficacy of NK cell significantly decreases when the DS increases from 18% to 26%. As a result, it can be seen that the DS needs to be controlled to be greater than 7% and less than 26% in order to improve the cancer cell killing efficiency of NK cell through the HA-PEG-DSPE polymer compound bound to NK cell.

Referring to FIG. 54, the NK cell coating efficacy according to Log P for HA-PEG-DSPE having various PEG lengths and various DSs is summarized and shown. The Log P value was calculated by <Equation 1> described above. As can be seen in FIG. 8, it can be seen that PEG having a length of 2 k and HA-PEG-DSPE having the DS of 18% have significantly higher NK cell coating efficacy compared to other HA-PEG-DSPEs.

### Experimental Example 3-3: Analysis of Effect of Polymer Compound on NK Cell

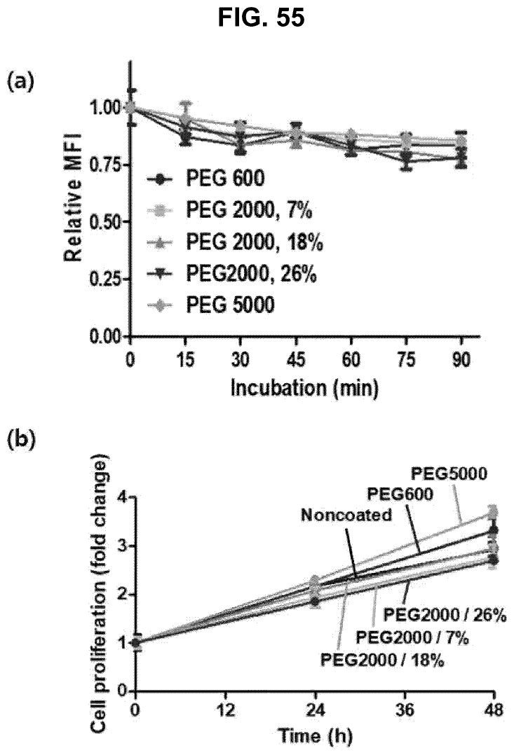

FIG. 55 is a graph showing a coating sustainability and cell proliferation ability of cells in which polymer compounds according to experimental examples of the present invention are bound to NK cells.

Referring to (a) of FIG. 55, the coating sustainability of HA-PEG-DSPE in cells in which various HA-PEG-DSPEs are bound to NK cells is shown. Specifically, it was measured using HA-PEG-DSPE using PEG 600, PEG 2000 (7%), PEG 2000 (18%), PEG 2000 (26%), and PEG 5000 m, and the coating sustainability was confirmed through a change in MFI value according to an incubation time (min). As can be seen in (a) of FIG. 55, it can be confirmed that all of different HA-PEG-DSPEs have a similar coating sustainability.

Referring to (b) of FIG. 55, the cell proliferation ability (fold change) of cells in which various HA-PEG-DSPEs are bound to NK cells is shown. Specifically, it was measuring using HA-PEG-DSPE using PEG 600, PEG 2000 (7%), PEG 2000 (18%), PEG 2000 (26%), and PEG 5000. In addition, the cell proliferation ability of NK cell to which HA-PEG-DSPE is not bound was also measured as a control. As can be seen in (b) of FIG. 55, it can be confirmed that all of different HA-PEG-DSPEs have a similar cell proliferation ability.

In other words, when HA-PEG-DSPE is bound to NK cell, it can be seen that a change in PEG length and lipid content does not substantially affect the coating sustainability and cell proliferation ability.

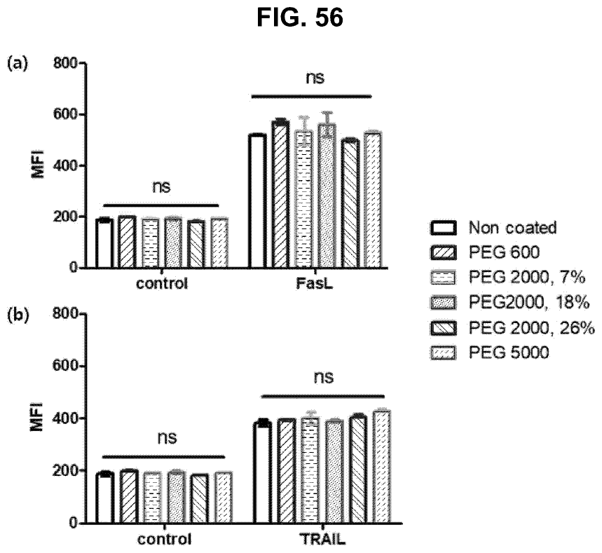

FIG. 56 is a graph for describing an effect of polymer compounds on a ligand of NK cell according to experimental examples of the present invention.

Referring to FIG. 56, in order to evaluate whether TRAIL and FasL, which are two representative cell membrane ligands required for cancer cell recognition by NK cell, function normally, the result of making an MFI analysis by flow cytometry after treating cells in which various HA-PEG-DSPEs are bound to NK cells with TRAIL antibody and FasL antibody, and then detecting TRAIL and FasL present on the surface of each cell is shown. Specifically, it was measured using HA-PEG-DSPE using PEG 600, PEG 2000 (7%), PEG 2000 (18%), PEG 2000 (26%), and PEG 5000. In addition, NK cell (non-coated) to which HA-PEG-DSPE is not bound was also measured as a control group. Furthermore, (a) of FIG. 56 shows the results for FasL, and (b) of FIG. 56 shows the results for TRAIL.

As can be seen in FIG. 56, it can be confirmed that TRAIL and FasL ligands normally function in all NK cells to which different HA-PEG-DSPEs are bound. In other words, when HA-PEG-DSPE is bound to NK cell, it can be confirmed that a change in PEG length and lipid content does not affect TRAIL and FasL ligands, which are NK cell-specific ligands.

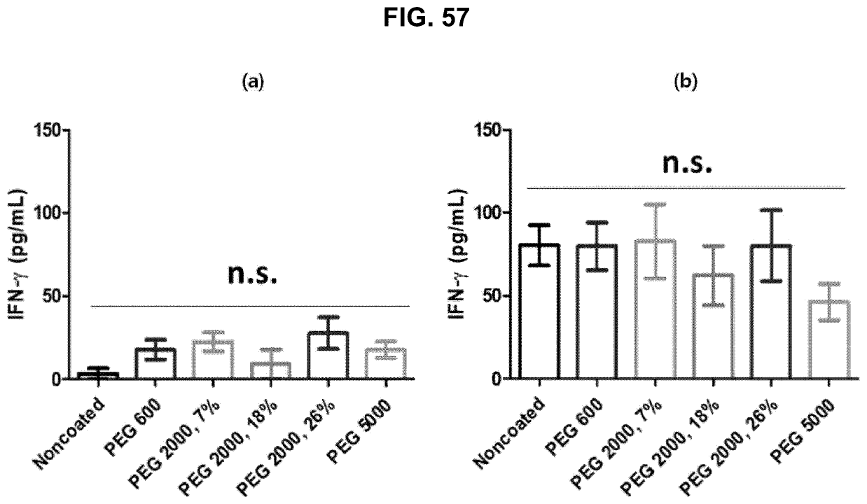

FIG. 57 is a graph for describing an effect of polymer compounds on cytokine secretion of NK cell according to experimental examples of the present invention.

Referring to FIG. 57, in order to evaluate whether the secretion of cytokine (IFN-Y HA-PEG-DSPE), which is a representative material of NK cell for cancer cell death, normally functions, the amount (pg/mL) of cytokine (IFN-Y) secreted from cells bound to NK cell is quantified and shown. Specifically, HA-PEG-DSPE using PEG 600, PEG 2000 (7%), PEG 2000 (18%), PEG 2000 (26%), and PEG 5000 was used. In addition, NK cell (non-coated) to which HA-PEG-DSPE is not bound was also measured as a control group. Furthermore, (a) of FIG. 57 shows a state in which LPS, which is a substance for promoting cytokine secretion, is not treated, and (b) of FIG. 57 shows a state in which LPS is treated.

As can be seen in FIG. 57, it can be confirmed that the secretion of cytokine (IFN-Y) normally functions in all NK cells to which different HA-PEG-DSPEs are bound. In other words, when HA-PEG-DSPE is bound to NK cell, it can be confirmed that a change in PEG length and lipid content does not affect cytokine (IFN-Y) secretion, which is one of the unique functions of NK cell.

### Experimental Example 3-4: Analysis of Cancer Cell Targeting Ability of NK Cell to which Polymer Compound is Bound

NK cell was stained with calcein AM (green reagent) and target cell was stained with cell tracker red (red reagent) and then bound to various HA-PEG-DSPEs. After that, the bound cells were co-incubated with target cell at a ratio of 1:1 for 30 minutes, and then an effector cell to target cell cluster ratio (E:T cluster) caught in both FITC and APC regions was detected by flow cytometry.

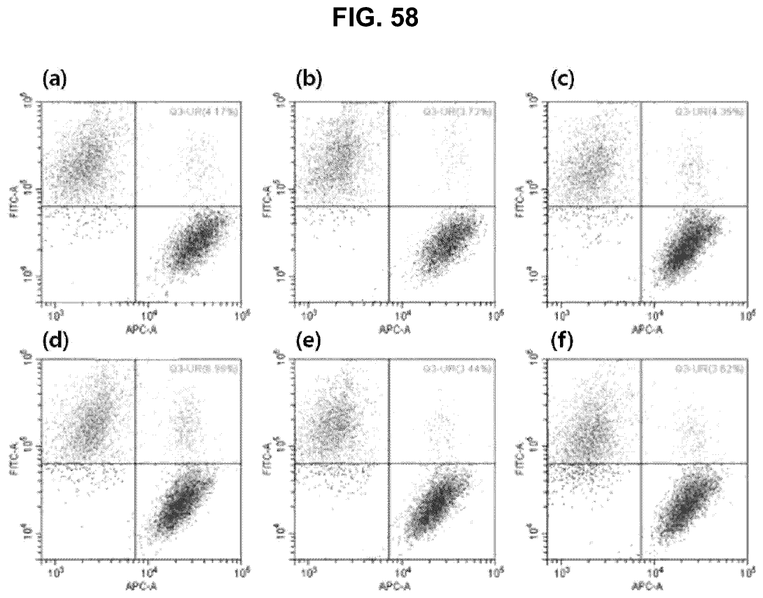

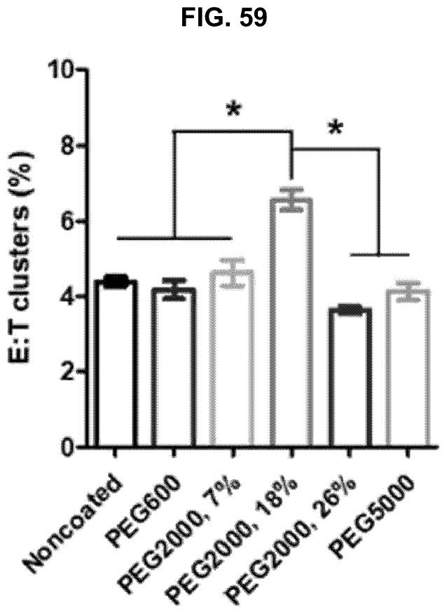

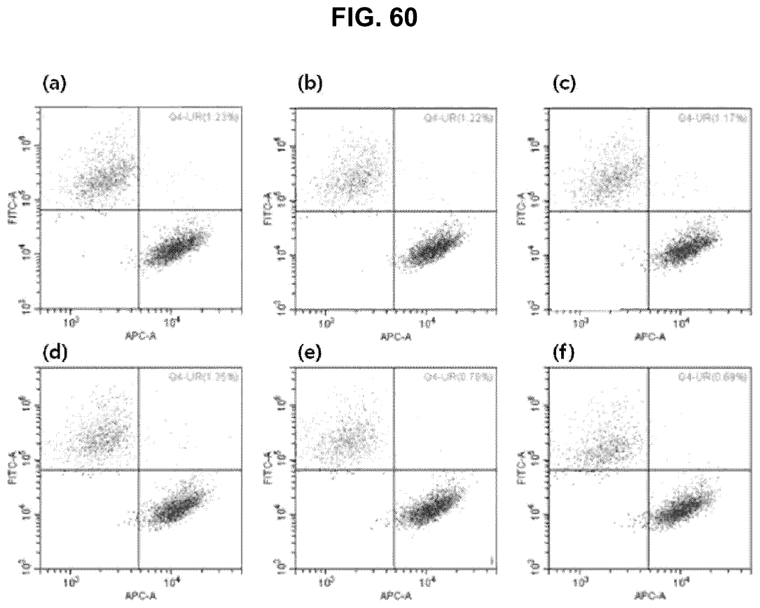

FIG. 58 is a view for confirming a targeting ability of NK cell, to which polymer compounds are bound, for triple negative breast cancer cells according to experimental examples of the present invention, and FIG. 59 is a graph quantifying an E:T cluster ratio measured in FIG. 58.

Referring to FIGS. 58 and 59, E:T cluster was detected by the method according to Experimental Example 4 described above, but triple negative breast cancer cells (MDA-MB-231) with CD44 overexpressed were used as target cell. More specifically, (a) of FIG. 58 shows a result for NK cell to which HA-PEG-DSPE is not bound, (b) of FIG. 58 shows a result for NK cell to which HA-PEG-DSPE using PEG 600 is bound, (c) of FIG. 58 shows a result for NK cell to which HA-PEG-DSPE using PEG 2000 (7%) is bound, (d) of FIG. 58 shows a result for NK cell to which HA-PEG-DSPE using PEG 2000 (18%) is bound, (e) of FIG. 58 shows a result for NK cell to which HA-PEG-DSPE using PEG 2000 (26%) is bound, and (f) of FIG. 58 shows a result for NK cell to which HA-PEG-DSPE using PEG 5000 is bound.

As can be seen in FIGS. 58 and 59, in the case of HA-PEG-DSPE using PEG 2000 (18%), it can be confirmed that the target ability for triple negative breast cancer cells (MDA-MB-231) is significantly higher compared to other HA-PEG-DSPEs.

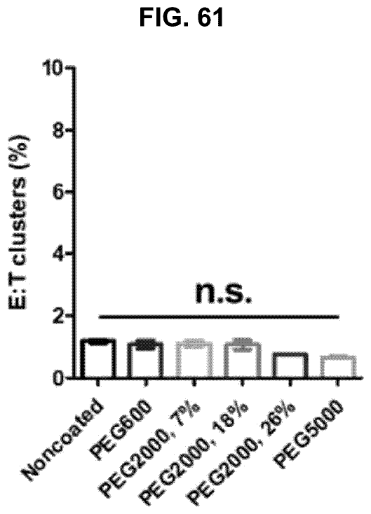

FIG. 60 is a view for confirming a targeting ability of NK cell, to which polymer compounds are bound, for liver cancer cells according to experimental examples of the present invention, and FIG. 61 is a graph quantifying an E:T cluster ratio measured in FIG. 60.

Referring to FIGS. 60 and 61, E:T cluster was detected by the method according to Experimental Example 4 described above, but liver cancer cells (HepG2) with CD44 hardly expressed were used as target cell. More specifically, (a) of FIG. 60 shows a result for NK cell to which HA-PEG-DSPE is not bound, (b) of FIG. 60 shows a result for NK cell to which HA-PEG-DSPE using PEG 600 is bound, (c) of FIG. 60 shows a result for NK cell to which HA-PEG-DSPE using PEG 2000 (7%) is bound, (d) of FIG. 60 shows a result for NK cell to which HA-PEG-DSPE using PEG 2000 (18%) is bound, (e) of FIG. 60 shows a result for NK cell to which HA-PEG-DSPE using PEG 2000 (26%) is bound, and (f) of FIG. 60 shows a result for NK cell to which HA-PEG-DSPE using PEG 5000 is bound.

As can be seen in FIGS. 60 and 61, it can be confirmed that all of different HA-PEG-DSPEs have substantially no targeting ability for liver cancer cells. In other words, it can be seen that HA-PEG-DSPE selectively recognizes cancer cells with CD44 overexpressed.

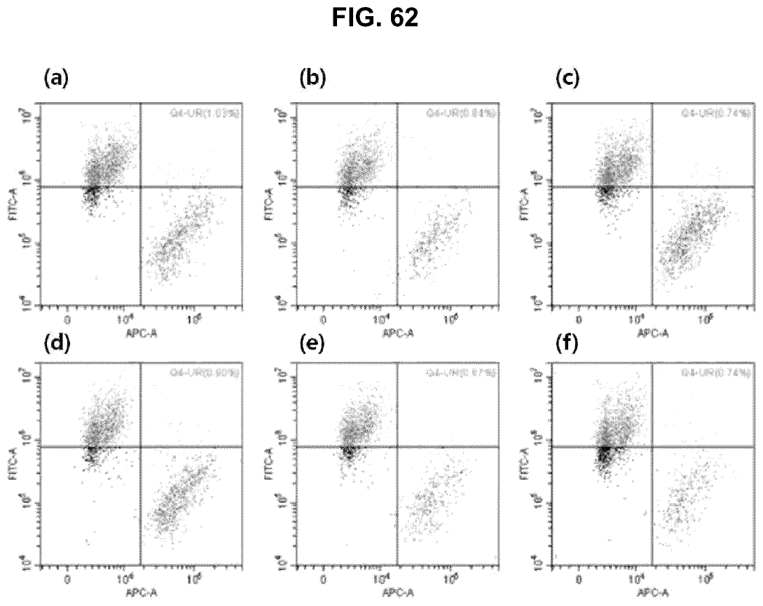

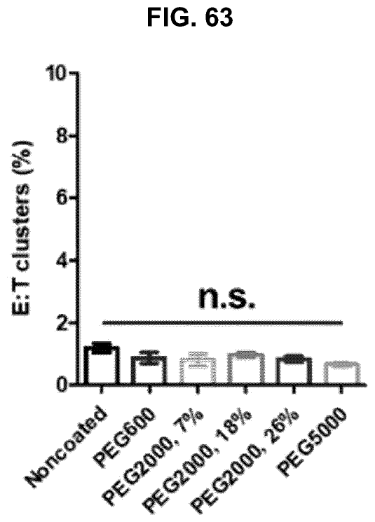

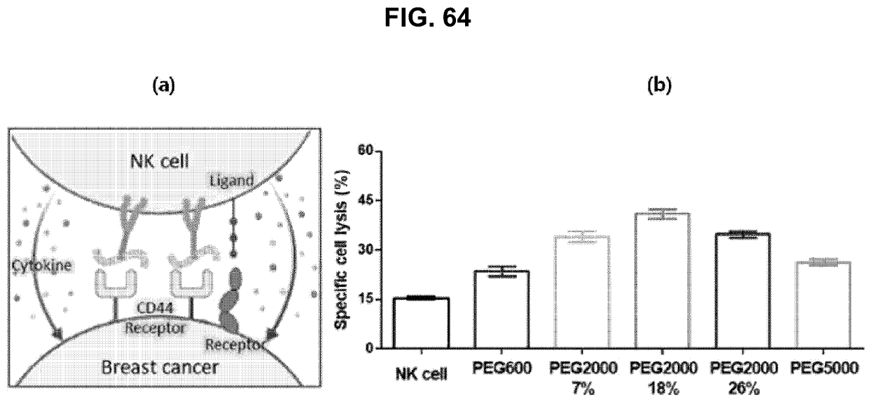

FIG. 62 is a view for confirming a targeting ability of NK cell, to which polymer compounds are bound, for fibroblasts according to experimental examples of the present invention, and FIG. 63 is a graph quantifying an E:T cluster ratio measured in FIG. 62.

Referring to FIGS. 62 and 63, E:T cluster was detected by the method according to Experimental Example 4 described above, but fibroblasts, which are normal cells, were used as target cell. More specifically, (a) of FIG. 62 shows a result for NK cell to which HA-PEG-DSPE is not bound, (b) of FIG. 62 shows a result for NK cell to which HA-PEG-DSPE using PEG 600 is bound, (c) of FIG. 62 shows a result for NK cell to which HA-PEG-DSPE using PEG 2000 (7%) is bound, (d) of FIG. 62 shows a result for NK cell to which HA-PEG-DSPE using PEG 2000 (18%) is bound, (e) of FIG. 62 shows a result for NK cell to which HA-PEG-DSPE using PEG 2000 (26%) is bound, and (f) of FIG. 62 shows a result for NK cell to which HA-PEG-DSPE using PEG 5000 is bound.

As can be seen in FIGS. 62 and 63, it can be confirmed that all of different HA-PEG-DSPEs have substantially no targeting ability for fibroblasts. In other words, it can be seen again that HA-PEG-DSPE selectively recognizes cancer cells with CD44 overexpressed.

### Experimental Example 3-5: Analysis of Cancer Cell Killing Ability of NK Cell to which Polymer Compound is Bound

A HA-PEG-DSPE polymer compound was dissolved in MEM alpha at a concentration of 1.0 mg/mL, and 6×10⁵ NK92-mi NK cells were evenly mixed with 120 μL of composite material solution. The surface of NK cell was modified (HA-PEG-DSPE bound to NK cell) at room temperature for 30 minutes and then washed twice with MEM alpha.

6×10⁴ target cells were stained with 60 μL of 15 μM calcein-AM solution at 37° C. for 30 minutes, and then washed twice with HDMEM. The NK cell of which the surface is completely modified and the target cell which is completely stained were added together at a ratio of 10:1 to 96-well plate and incubated at 37° C. for four hours, and then a supernatant was obtained and a fluorescence intensity was measured at 480/535 mm.

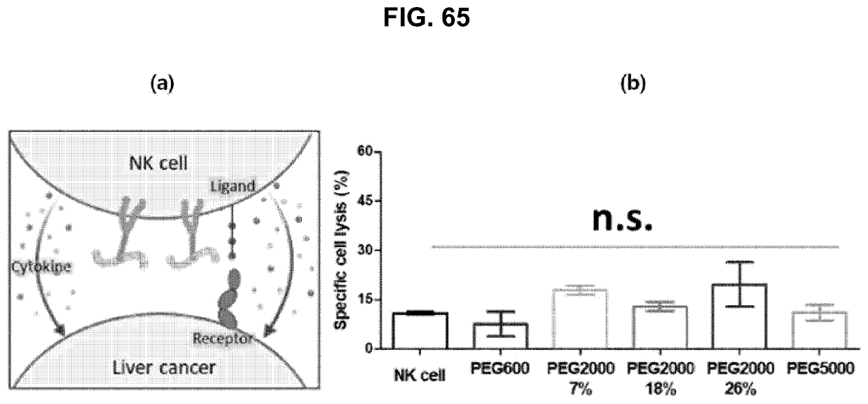

FIG. 64 is a view for describing a killing ability of NK cell, to which polymer compounds are bound, for triple negative breast cancer cells according to experimental examples of the present invention.

Referring to FIG. 64, a lysis ability (specific cell lysis, %) of target cell was measured by the method according to Experimental Example 3-5 described above, and a triple negative breast cancer cell (MDA-MB-231) with CD44 overexpressed was used as the target cell. In addition, HA-PEG-DSPE using PEG 600, PEG 2000 (7%), PEG 2000 (18%), PEG 2000 (26%), and PEG 5000 was used as various polymer compounds, and the lysis ability of NK cell to which HA-PEG-DSPE was not bound was also measured. (a) of FIG. 64 shows a schematic view of a process in which the NK cell to which the polymer compound is bound kills triple negative breast cancer cells, and (b) of FIG. 64 quantitatively shows a degree of lysis for triple negative breast cancer cells (MDA-MB-231).

As can be seen in FIG. 64, it can be confirmed that a degree of lysis of NK cell to which HA-PEG-DSPE is bound is improved for the triple negative breast cancer cells compared to NK cell to which HA-PEG-DSPE is not bound. In particular, in the case of HA-PEG-DSPE using PEG 2000 (18%), it can be confirmed that a degree of lysis for triple negative breast cancer cells (MDA-MB-231) is significantly improved.

FIG. 65 is a view for describing a killing ability of NK cell, to which polymer compounds are bound, for liver cancer cells according to experimental examples of the present invention.

Referring to FIG. 65, a lysis ability (specific cell lysis, %) of target cell was measured by the method according to Experimental Example 3-5 described above, and a liver cancer cell (HepG2) with CD44 hardly expressed was used as the target cell. In addition, HA-PEG-DSPE using PEG 600, PEG 2000 (7%), PEG 2000 (18%), PEG 2000 (26%), and PEG 5000 was used as various polymer compounds, and the lysis ability of NK cell to which HA-PEG-DSPE was not bound was also measured. (a) of FIG. 65 shows a schematic view of a process in which the NK cell to which the polymer compound is bound kills liver cancer cells, and (b) of FIG. 65 quantitatively shows a degree of lysis for liver cancer cells (HepG2).

As can be seen in FIG. 65, it can be confirmed that NK cell to which HA-PEG-DSPE is bound has substantially no difference in killing ability for liver cancer cells compared to NK cell to which HA-PEG-DSPE is not bound. In other words, it can be seen that NK cell to which HA-PEG-DSPE is bound selectively kills cancer cells with CD44 overexpressed.

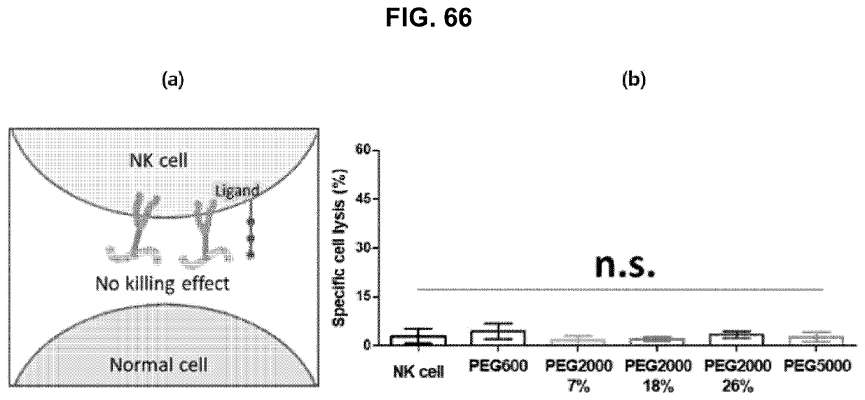

FIG. 66 is a view for describing a killing ability of NK cell, to which polymer compounds are bound, for fibroblasts according to experimental examples of the present invention.

Referring to FIG. 66, a lysis ability (specific cell lysis, %) of target cell was measured by the method according to Experimental Example 3-5 described above, and fibroblasts, which are normal cells, were used as target cell. In addition, HA-PEG-DSPE using PEG 600, PEG 2000 (7%), PEG 2000 (18%), PEG 2000 (26%), and PEG 5000 was used as various polymer compounds, and the lysis ability of NK cell to which HA-PEG-DSPE was not bound was also measured. (a) of FIG. 66 shows a schematic view of a process in which the NK cell to which the polymer compound is bound reacts with a fibroblast (normal cell), and (b) of FIG. 66 quantitatively shows a degree of lysis for a fibroblast.

As can be seen in FIG. 66, it can be confirmed that both NK cell to which HA-PEG-DSPE is bound and NK cell to which HA-PEG-DSPE is not bound do not lyse fibroblasts, which are normal cells. In other words, it can be seen again that NK cell to which HA-PEG-DSPE is bound selectively kills cancer cells with CD44 overexpressed.

Although the present invention has been described in detail using preferred embodiments, the scope of the present invention is not limited to specific embodiments and should be interpreted by the appended claims. In addition, it should be understood by those skilled in the art that many modifications and variations are possible without departing from the scope of the present invention.

### INDUSTRIAL APPLICABILITY

The present invention may be used in the medical industry.

1. A polymer compound comprising a hydrophobic moiety binding to a natural killer (NK) cell, a cancer cell recognition moiety, and a linker, wherein
the hydrophobic moiety is bound to one end of the linker and the cancer cell recognition moiety is bound to the other end of the linker, so as to recognize NK cells and cancer cells, in which
the cancer cell recognition moiety includes hyaluronic acid.

2. The polymer compound of claim 1, wherein the cancer cell recognition moiety selectively recognizes solid cancer cells including CD44.

3. The polymer compound of claim 1, wherein the cancer cell recognition moiety recognizes pancreatic cancer cells (MIA PaCa-2), triple negative breast cancer cells (MDA-MB-231), and colorectal cancer cells (HCT-116).

4. The polymer compound of claim 1, wherein the polymer compound bound to the NK cell through the hydrophobic moiety is bound to the cancer cell through the cancer cell recognition moiety, and
promotes a secretion of cytotoxic granules and cytokines from the NK cell to kill the cancer cell.

5. The polymer compound of claim 1, wherein the hydrophobic moiety includes a lipid, and is bound to a surface of the NK cell through a hydrophobic interaction via the lipid.

6. The polymer compound of claim 1, wherein the linker prevents the polymer compound bound to the NK cell from being subjected to endocytosis into the NK cell.

7. The polymer compound of claim 1, wherein the linker includes polyethylene glycol (PEG).

8. The polymer compound of claim 1, wherein the polymer compound bound to the NK cell is removed from the NK cell within 36 hours.

9. The polymer compound of claim 8, wherein the polymer compound bound to the NK cell is naturally removed from the NK cell without an external physical and chemical intervention.

10. A polymer compound comprising a hydrophobic moiety binding to a natural killer (NK) cell, a cancer cell recognition moiety, and a linker, wherein
the hydrophobic moiety is bound to one end of the linker and the cancer cell recognition moiety is bound to the other end of the linker, so as to recognize NK cells and cancer cells, in which
the cancer cell recognition moiety includes hyaluronic acid, and
the hydrophobic moiety includes 1,2-distearoyl-sn-glycero-3-phosphorylethanolamine (DSPE).

11. The polymer compound of claim 10, wherein a peak of the hydrophobic moiety is not observed in a range of 6 to 9 ppm as a result of proton NMR analysis.

12. The polymer compound of claim 10, wherein a binding efficiency and a binding persistence for the NK cells are improved as compared with a polymer compound including 1,2-dimyristoyl-sn-glycero-3-phosphoethanolamine (DMPE) as the hydrophobic moiety and a polymer compound including cholesterol as the hydrophobic moiety.

13. The polymer compound of claim 10, wherein the cancer cell recognition moiety selectively recognizes solid cancer cells including CD44.

14. The polymer compound of claim 10, wherein the cancer cell recognition moiety recognizes pancreatic cancer cells (MIA PaCa-2), triple negative breast cancer cells (MDA-MB-231), and colorectal cancer cells (HCT-116).

15. The polymer compound of claim 10, wherein the linker includes polyethylene glycol (PEG).

16. A polymer compound comprising a hydrophobic moiety binding to a natural killer (NK) cell, a cancer cell recognition moiety, and a linker, wherein
the hydrophobic moiety is bound to one end of the linker and the cancer cell recognition moiety is bound to the other end of the linker and recognizes NK cells and cancer cells, in which
the cancer cell recognition moiety includes hyaluronic acid, and
a length of the linker and a content of the hydrophobic moiety are controlled to improve killing efficiency of solid cancer cells including CD44 through the NK cells.

17. The polymer compound of claim 16, wherein the linker includes polyethylene glycol (PEG).

18. The polymer compound of claim 17, wherein the linker has a length greater than 0.6 k and less than 5 k.

19. The polymer compound of claim 16, wherein the hydrophobic moiety includes 1,2-distearoyl-sn-glycero-3-phosphorylethanolamine (DSPE).

20. The polymer compound of claim 19, wherein a degree of substitution (DS) defined as a value obtained by calculating an amount of the hydrophobic moiety linked to the cancer cell recognition moiety through an NMR analysis is greater than 7% and less than 26%.
